(19) United States
(12) Patent Application Publication
(10) Pub. No.: US 2013/0254934 A1
(43) Pub. Date: Sep. 26, 2013

(54) DISEASE-RESISTANT PLANT AND METHOD FOR PREPARING THE SAME

(71) Applicants: Takeshi Nakano, Saitama (JP); Hideo Nakashita, Saitama (JP); Tadao Asami, Saitama (JP); Michiko Yasuda, Saitama (JP); Ayumi Yamagami, Saitama (JP)

(72) Inventors: Takeshi Nakano, Saitama (JP); Hideo Nakashita, Saitama (JP); Tadao Asami, Saitama (JP); Michiko Yasuda, Saitama (JP); Ayumi Yamagami, Saitama (JP)

(21) Appl. No.: 13/992,485

(22) PCT Filed: Dec. 9, 2011
(86) PCT No.: PCT/JP2011/078559

§ 371 Date: Jun. 7, 2013

(30) Foreign Application Priority Data
Dec. 10, 2010 (JP) 2010-275925

(51) Int. Cl. C12N 15/82

(52) U.S. Cl. CPC C12N 15/8281
USPC 800/279; 800/301

(57) ABSTRACT

It is intended to identify a novel gene involved in the induction of pathogen resistance by brassinosteroid and to prepare a disease-resistant plant with enhanced disease resistance by introducing the gene into plant cells of interest. Pathogen-resistant brassinosteroid mutants are isolated, and four genes obtained on the basis of the analysis of causative genes of the mutants were introduced into plant cells of interest to prepare transgenic plants.

### TECHNICAL FIELD

The present invention relates to a disease-resistant plant and a method for preparing the same.

### BACKGROUND ART

Microbial infection is inevitable for plants and usually causes serious stress to plant bodies. Against such microbial infection, particularly, pathogenic infection, plants have evolved their own protective or defensive systems, in addition to morphological adaptation. Specifically, the primary response of each plant to the pathogenic infection involves the specific recognition of the pathogens and the rapid induction of cell death (hypersensitive cell death) of infected cells to eliminate the pathogens together with the infected cells (Non Patent Literature 1). The secondary response of the plant is the induction of pathogen resistance called systemic acquired resistance (SAR), which is triggered by the hypersensitive cell death, in order to protect the plant body from further attacks by the pathogens (Non Patent Literatures 2 and 3). SAR has been confirmed in many plants and confers resistance to various plant pathogens to the uninfected portions of plants (Non Patent Literatures 4 and 5). Salicylic acid has been identified as a signaling factor inducing this SAR, in dicotyledon such as Arabidopsis thaliana and tobacco (Non Patent Literatures 6 and 7). If other signaling factors inducing SAR are identified and the intracellular signals of plants can be controlled, disease resistance can be conferred to plant bodies without being mediated by hypersensitive cell death. Unfortunately, much remains to be revealed about this SAR-inducing signaling mechanism mediated by salicylic acid, and the whole picture of the mechanism has not yet been clarified.

### CITATION LIST

### Non Patent Literature

Non Patent Literature 1: Ross A. F. (1961) Virology, 14: 340-358
Non Patent Literature 2: Kuc J. (1982) Bioscience, 32: 854-860
Non Patent Literature 3: McIntyre J. L. et al., (1981) Phytopathology, 71: 297-301
Non Patent Literature 4: Chester K S., (1933) Q. Rev. Biol., 8: 275-324
Non Patent Literature 5: Durner J. et al., (1997) Trends in Plant Sci., 2: 266-274
Non Patent Literature 6: Delaney T P. et al., (1994) Science, 266: 1247-1250
Non Patent Literature 7: Gaffney T. et al., (1993) Science, 261: 754-756

### SUMMARY OF INVENTION

### Technical Problem

The present inventors have newly found the induction of disease resistance by brassinosteroid treatment (brassinosteroid-mediated disease resistance; hereinafter, referred to as “BDR”) which is different from the salicylic acid-induced disease resistance described above (Nakashita H. et al., 2003, The Plant Jour., 33: 887-898). Brassinosteroid (hereinafter, referred to as “BR”) is a phytohormone that is involved in the regulation of plant growth, photomorphogenesis, the control of vascular bundle formation, the functional regulation of chloroplasts, etc. (Azpiroz R. et al., 1988, Plant Cell, 10: 219-230; Clouse S. & Sasse J., 1998, Annu. Rev. Plant Physiol. Plant Mol. Biol., 49: 427-451; Mandava N., 1988, Annu. Rev. Plant Physiol. Plant Mol. Biol., 39: 23-52; and Sakurai A. et al., 1999, Brassinosteroids, Steroidal Plant Hormones, Tokyo: Springer). The involvement of BR in the induction of disease resistance has been found for the first time. Accordingly, BR has received attention as a novel candidate of a plant disease resistance-enhancing agent. However, a method of continuously applying expensive BR to plant bodies all the time is disadvantageously unrealistic for practice at agricultural sites. Meanwhile, the induction of BDR by BR treatment indicates that an intracellular signaling pathway activated by BR (hereinafter, referred to as a “BR intracellular signaling pathway”) is presumably able to induce disease resistance. Unfortunately, much remains to be revealed about the BR intracellular signaling pathway. In addition, a signaling factor that functions in this pathway (hereinafter, referred to as a “BR intracellular signaling factor”) has not yet been fully identified. Furthermore, a transcriptional factor BIL1, which has already been identified as such a BR intracellular signaling molecule, has been shown to be unable to induce disease resistance. These facts suggested that the BR intracellular signaling pathway may not be related to the induction of BDR.

An object of the present invention is to identify a novel gene involved in the induction of BDR and to prepare a disease-resistant plant with enhanced disease resistance by introducing the gene into plant cells of interest.

### Solution to Problem

To attain the object, the present inventors have predicted that the BR intracellular signaling pathway is separated in midstream into branches, one or some of which control the induction of BDR. Thus, on the basis of this hypothesis, the present inventors have tested the disease resistance of Arabidopsis thaliana mutants presumably associated with the signaling pathway, and successfully isolated mutants bil5-1D and bil4-1D that exhibit infection resistance to a tomato opportunistic bacterium Pseudomonas syringae 3000. Results of analyzing these mutants demonstrated that this trait results from a gain-of-function mutation in BIL5 gene or BIL4 gene. The involvement of a BIL5 gene product (BIL5) and a BIL4 gene product (BIL4) (in the present specification, hereinafter, the simple description of, for example, “BIL5”, means the “BIL5 protein” as a rule, whereas a gene encoding BIL5 is indicated by BIL5 plus the term “gene”, as in the “BIL5 gene”) in plant disease resistance is new findings. In view of this result, the wild-type BIL5 gene or the wild-type BIL4 gene was overexpressed in a wild-type Arabidopsis thaliana strain. As a result, the present inventors have found that plant disease resistance can be induced in the plant bodies. The present inventors have also found that novel BNX1 gene, which is a putative paralog of the BIL5 gene, and BIL6 gene also produce similar effects when overexpressed in plant cells. The present invention is based on these new findings and provides the followings:
(1) A method for preparing a disease-resistant plant, comprising the step of introducing into a plant of interest at least one nucleic acid expression system incorporating, in an expressible state, a nucleic acid encoding any of the following polypeptides (a) to (c) or a fragment thereof having plant disease resistance-enhancing activity:

(a) a polypeptide consisting of the amino acid sequence represented by SEQ ID NO: 1 or 2,

(b) a polypeptide consisting of an amino acid sequence derived from the amino acid sequence represented by SEQ ID NO: 1 or 2 by the deletion, substitution, or addition of one or several amino acids and having plant disease resistance-enhancing activity, and

(c) a polypeptide consisting of an amino acid sequence having 40% or more identity to the amino acid sequence represented by SEQ ID NO: 1 or 2 and having plant disease resistance-enhancing activity.
(2) The preparation method according to (1), wherein the nucleic acid is a nucleic acid consisting of the nucleotide sequence represented by SEQ ID NO: 3 or 4, or a nucleic acid that hybridizes under stringent conditions to the nucleic acid consisting of the nucleotide sequence represented by SEQ ID NO: 3 or 4 and encodes a polypeptide having plant disease resistance-enhancing activity.
(3) A method for preparing a disease-resistant plant, comprising the step of introducing at least one nucleic acid expression system incorporating, in an expressible state, a nucleic acid encoding any of the following polypeptides (a) to (c) or a fragment thereof having plant disease resistance-enhancing activity into a plant of interest:

(a) a polypeptide consisting of the amino acid sequence represented by SEQ ID NO: 11 or 12,

(b) a polypeptide consisting of an amino acid sequence derived from the amino acid sequence represented by SEQ ID NO: 11 or 12 by the deletion, substitution, or addition of one or several amino acids and having plant disease resistance-enhancing activity, and

(c) a polypeptide consisting of an amino acid sequence having 40% or more identity to the amino acid sequence represented by SEQ ID NO: 11 or 12 and having plant disease resistance-enhancing activity.
(4) The preparation method according to (3), wherein the nucleic acid is a nucleic acid consisting of the nucleotide sequence represented by SEQ ID NO: 13 or 14, or a nucleic acid that hybridizes under stringent conditions to the nucleic acid consisting of the nucleotide sequence represented by SEQ ID NO: 13 or 14 and encodes a polypeptide having plant disease resistance-enhancing activity.
(5) The preparation method according to any of (1) to (4), wherein the nucleic acid expression system overexpresses the nucleic acid incorporated therein.
(6) The preparation method according to (5), wherein the nucleic acid expression system constitutively expresses the nucleic acid incorporated therein.
(7) The preparation method according to (5), wherein the nucleic acid expression system inducibly expresses the nucleic acid incorporated therein.
(8) The preparation method according to any of (1) to (7), wherein the nucleic acid expression system is an expression vector.
(9) A disease-resistant plant obtained by a preparation method according to any of (1) to (8), or progeny thereof.
(10) A disease-resistant plant comprising at least one exogenous nucleic acid expression system incorporating, in an expressible state, a nucleic acid encoding any of the following polypeptides (a) to (c) or a fragment thereof having plant disease resistance-enhancing activity:

(a) a polypeptide consisting of the amino acid sequence represented by SEQ ID NO: 1 or 2,

(b) a polypeptide consisting of an amino acid sequence derived from the amino acid sequence represented by SEQ ID NO: 1 or 2 by the deletion, substitution, or addition of one or several amino acids and having plant disease resistance-enhancing activity, and

(c) a polypeptide consisting of an amino acid sequence having 40% or more identity to the amino acid sequence represented by SEQ ID NO: 1 or 2 and having plant disease resistance-enhancing activity.
(11) A disease-resistant plant comprising at least one exogenous nucleic acid expression system incorporating, in an expressible state, a nucleic acid encoding any of the following polypeptides (a) to (c) or a fragment thereof having plant disease resistance-enhancing activity:

(a) a polypeptide consisting of the amino acid sequence represented by SEQ ID NO: 11 or 12,

(b) a polypeptide consisting of an amino acid sequence derived from the amino acid sequence represented by SEQ ID NO: 11 or 12 by the deletion, substitution, or addition of one or several amino acids and having plant disease resistance-enhancing activity, and

(c) a polypeptide consisting of an amino acid sequence having 40% or more identity to the amino acid sequence represented by SEQ ID NO: 11 or 12 and having plant disease resistance-enhancing activity.

The present specification encompasses the contents described in the specification and/or drawings of Japanese Patent Application No. 2010-275925 on which the priority of the present application is based.

### Advantageous Effects of Invention

The method for preparing a disease-resistant plant according to the present invention can confer or enhance disease resistance to or in a desired plant.

The disease-resistant plant progeny of the present invention can provide subsequent plant generations with the acquired disease resistance.

### BRIEF DESCRIPTION OF DRAWINGS

FIG. 1 is a diagram showing the induction of pathogen resistance marker PR1 gene expression in a gain-of-function mutant bil5-1D of Arabidopsis thaliana. Expression levels of PR1 gene in BR-unsupplemented (−) and BR-supplemented (+) samples of each strain are shown.

FIG. 2 is a diagram showing the induction of PR1 gene expression in a double overexpression strain of the wild-type BIL5 gene and the wild-type BNX1 gene (BIL5-OX1×BNX1-OX1 strain).

FIG. 3 is a diagram showing the acquisition of pathogen resistance to a tobacco bacterial pathogen (Pseudomonas syringae pv. tabaci: Pst) by the bil5-1D strain, etc.

FIG. 4 is a diagram showing the amount of free salicylic acid in the bil5-1D strain, etc.

FIG. 5 is a diagram showing the acquisition of pathogen resistance by the BIL5-OX1×BNX1-OX1 strain, etc. Pst represents the tobacco bacterial pathogen.

FIG. 6 is a diagram showing the amount of free salicylic acid in the BIL5-OX1×BNX1-0X1 strain, etc.

FIG. 7 is a diagram showing the induction of PR1 gene (A) or PR5 gene (B) expression in a wild-type BIL4 gene-overexpressing strain (BIL4-OX1).

FIG. 8 is a diagram showing the induction of PR1 gene (A) or PR5 gene (B) expression in two wild-type BIL6 gene-overexpressing strains (BIL6-OX1 and BIL6-OX2).

### DESCRIPTION OF EMBODIMENTS

### 1. Method for Preparing Disease-Resistant Plant

The first embodiment of the present invention relates to a method for preparing a disease-resistant plant. The preparation method of the present invention comprises the step of introducing a nucleic acid expression system into a plant of interest (nucleic acid expression system introduction step). Specifically, the method for preparing a disease-resistant plant according to this embodiment is a method for preparing a transgenic plant having disease resistance. The preparation method of the present invention can confer disease resistance to the plant. Thus, the method of the present invention also serves as a method for conferring disease resistance to a plant.

In the present specification, the “transgenic plant” refers to a transformed plant that has acquired disease resistance by introduction of the nucleic acid expression system.

<Nucleic Acid Expression System Introduction step>

The “nucleic acid expression system introduction step” is the step of introducing at least one nucleic acid expression system incorporating, in an expressible state, a nucleic acid encoding any of four plant disease resistance polypeptides found by the present inventors or a variant polypeptide thereof having plant disease resistance-enhancing activity, or a fragment of the polypeptide or the variant polypeptide having the activity into a plant cell of interest. This step transforms the desired plant cell. Hereinafter, this step will be described in detail.

### (1) Constitution

In the present invention, the “disease” or the “plant disease” refers to a disease in a plant caused by a pathogen. In this context, the “pathogen” refers to any of agents, such as viroids, viruses, phytoplasmas, bacteria, fungi (including yeasts, filamentous fungus, and basidiomycetes), slime molds, protozoans, or nematodes, which are infective to plants and bring about some pathological symptom to the plants through their infection. In the present invention, the pathogen preferably corresponds to, particularly, viroids, viruses, phytoplasmas, and bacteria. In the present invention, the “plant” corresponds to bryophytes, ferns, angiosperms, and gymnosperms. The angiosperms encompasses both dicotyledon and monocotyledon.

The “disease resistance” refers to the effect of preventing or suppressing the pathogenic infection or the onset of a pathological symptom caused thereby. This effect is controlled by the natural immune system of a plant. Thus, the “disease-resistant plant” means a plant with enhanced disease resistance and also means a plant with a potentiated natural immune system. In this context, the “disease-resistant plant” is not limited to plant bodies and encompasses all of the cells, tissues, and organs (embryos, meristems, seeds, shoots, roots, stems, leaves, and flowers) of plants having disease resistance.

The “four plant disease resistance polypeptides” (in the present specification, also referred to as “four proteins”, “four polypeptides”, or “four wild-type polypeptides”) specifically refer to wild-type BIL5, BNX1, BIL4, and BILE. The term “wild-type” refers to a protein that has the original functions of the protein and is typically encoded by alleles most commonly found among allele populations of the same type present in the natural world. The four proteins have all been shown to function in disease resistance for the first time as a result of studies conducted by the present inventors. Hereinafter, each protein will be described.

The protein “BIL5 (Brz-insentive-long hypocotyl 5)” is encoded by BIL5 gene. The BIL5 gene was identified as a causative gene of an Arabidopsis thaliana mutant bil5 having resistance to BR biosynthesis inhibitor; brassinazole (hereinafter, referred to as Brz), which specifically inhibits P450 monooxygenase; DWF4 in BR biosynthesis. The results of the studies conducted by the present inventors suggest that BIL5 is an intracellular signaling factor that has TIR (Toll/IL-1 receptor), NBS (nucleotide binding site), and LRR domains in this order and positively regulates plant development and disease resistance, downstream of BRI1 in the BR signaling pathway. The specific amino acid sequence of Arabidopsis thaliana-derived wild-type BIL5 is shown in SEQ ID NO: 1 (NCBI accession No. NM_{—}105051).

In the present invention, BIL5 encompasses not only the Arabidopsis thaliana-derived BIL5 (hereinafter, referred to as “Arabidopsis thaliana BIL5”) but orthologs of Arabidopsis thaliana BIL5 derived from other organism species. The orthologs of Arabidopsis thaliana BIL5 have 40% or more identity to the amino acid sequence of Arabidopsis thaliana BIL5 and also have plant disease resistance-enhancing activity similar thereto. Specific examples thereof include grape BIL5 (NCBI Gene-ID No. 100243035), Ricinus communis (castor bean) BIL5 (NCBI Gene-ID No. 8288150), Populus trichocarpa BIL5 (NCBI Gene-ID No. 7463263), Raphanus sativus BIL5 (NCBI accession No. CAZ40338), Brassica rapa BIL5 (NCBI accession No. ACP30636), tomato BIL5 (NCBI accession No. BABP01013461), and soybean BIL5 (NCBI Gene-ID No. GU967682).

The protein “BNX1 (Bil5-NeXt1)” was identified as a protein encoded by BNX1 gene located adjacent to the locus of the BIL5 gene on the genome of Arabidopsis thaliana. BNX1 has 70% identity to BIL5 at the amino acid level, suggesting its possibility of being a paralog of BIL5. The specific amino acid sequence of Arabidopsis thaliana-derived wild-type BNX1 is shown in SEQ ID NO: 2 (NCBI accession No. NM_{—}105052).

In the present invention, BNX1 encompasses not only the Arabidopsis thaliana-derived BNX1 (hereinafter, referred to as “Arabidopsis thaliana BNX1”) but orthologs of Arabidopsis thaliana BNX1 derived from other organism species. The orthologs of Arabidopsis thaliana BNX1 have 40% or more identity to the amino acid sequence of Arabidopsis thaliana BNX1 and also have plant disease resistance-enhancing activity similar thereto. Specific examples thereof include Oryza sativa BNX1 (NCBI Gene-ID No. 4326532), barley BNX1 (NCBI Gene-ID No. 606485), grape BNX1 (wine grape) (NCBI Gene-ID No. 100260274), Ricinus communis BNX1 (NCBI Gene-ID No. 8282489), Populus trichocarpa BNX1 (NCBI Gene-ID No. 7463263), Raphanus sativus BNX1 (NCBI accession No. CAZ40338), Brassica rapa BNX1 (NCBI accession No. ACP30636), (NCBI accession No. 5939404), and tomato BNX1 (NCBI accession No. AAP44392).

The protein “BIL4 (Brz-incentive-long hypocotyl 4)” is encoded by BIL4 gene. The BIL4 gene was identified as a causative gene of a semidominant Arabidopsis thaliana mutant bil4-1D having Brz resistance and a slender dwarf-like character as in the bil5 mutant. The results of the studies conducted by the present inventors suggest that BIL4 is a novel protein having a seven-transmembrane domain and is an intracellular signaling factor that is colocalized with BRI1 and positively regulates plant development and disease resistance, downstream of BRI1 in the BR signaling pathway. The specific amino acid sequence of Arabidopsis thaliana-derived wild-type BIL4, which is composed of 239 amino acids, is shown in SEQ ID NO: 11 (NCBI accession No. NP_{—}191890.1).

In the present invention, BIL4 encompasses not only the Arabidopsis thaliana-derived BIL4 (hereinafter, referred to as “Arabidopsis thaliana BIL4”) but orthologs of Arabidopsis thaliana BIL4 derived from other organism species. The orthologs of Arabidopsis thaliana BIL4 have 40% or more identity to the amino acid sequence of Arabidopsis thaliana BIL4 and also have plant disease resistance-enhancing activity similar thereto. Specific examples thereof include Oryza sativa BIL4 (NCBI Gene-ID Nos. NP_{—}001051246.2, NP_{—}001051551.1, and NP_{—}001059026.1) and Medicago truncatula BIL4 (NCBI accession No. XP_{—}003590585.1).

The protein “BIL6 (Brz-insentive-long hypocotyl 6)” is encoded by BIL6 gene. The BIL6 gene was identified as a gene whose highly expressing strains exhibit Brz resistance similar to that of the bil5 mutant or the bil4 mutant. BIL6, a mammalian natural immune system signaling factor belonging to the protein kinase family, is relatively highly homologous to the kinase domain of the IRAK which is a serine-threonine kinase. The expression of pathogen resistance marker PR1 and PR5 genes was activated in transformants highly expressing BIL6, suggesting that this factor positively regulates plant disease resistance, downstream of BRI1 in the BR signaling pathway. The specific amino acid sequence of Arabidopsis thaliana-derived wild-type BIL6, which is composed of 456 amino acids, is shown in SEQ ID NO: 12 (NCBI accession No. NP_{—}191271).

In the present invention, BIL6 encompasses not only the Arabidopsis thaliana-derived BIL6 (hereinafter, referred to as “Arabidopsis thaliana BIL6”) but orthologs of Arabidopsis thaliana BIL6 derived from other organism species. The orthologs of Arabidopsis thaliana BIL6 have 40% or more identity to the amino acid sequence of Arabidopsis thaliana BIL6 and also have plant disease resistance-enhancing activity similar thereto. Specific examples thereof include Oryza sativa BIL6 (NCBI Gene-ID No. NP_{—}001048538.2) and Medicago truncatula BIL6 (NCBI accession Nos. XP_{—}003591872.1 and AES85204.1).

The “variant polypeptide thereof having plant disease resistance-enhancing activity” refers to any of variant BIL5, BNX1, BIL4, and BIL6, which have been mutated from the wild-type BIL5, BNX1, BIL4, and BIL6, respectively. These variant proteins all have plant disease resistance-enhancing activity equal to or higher than that of their respective wild-type proteins. The variant polypeptide specifically refers to a polypeptide comprising an amino acid sequence derived from the amino acid sequence of each of the wild-type polypeptides by the deletion, substitution, or addition of one or several amino acids. Also, a polypeptide comprising an amino acid sequence derived from the amino acid sequence of each of the wild-type polypeptides by the modification such as post-translational methylation of one or several amino acids is encompassed in the variant polypeptide of the present invention. In this context, the term “several” refers to 2 to 20, 2 to 15, or 2 to 10, for example, 2 to 7, 2 to 5, 2 to 4, or 2 or 3 amino acids.

The “fragment thereof having the activity” refers to a polypeptide fragment of any of the four wild-type polypeptides, i.e., BIL5, BNX1, BIL4, and BIL6, or the variant polypeptide thereof having plant disease resistance-enhancing activity. This polypeptide fragment maintains plant disease resistance-enhancing activity equal to or higher than that of its parent polypeptide. The amino acid length of such a polypeptide fragment or its region in the full-length polypeptide differs depending on the functions, structure, etc., of the full-length polypeptide. Hence, it can be determined appropriately according to each protein. Usually, a polypeptide fragment containing a functional domain of the full-length protein in an undisrupted state is preferably used. In this context, the functional domain refers to a region that is responsible for a function unique to the protein or is indispensable in exerting the function. The functional domain corresponds to, for example, a nucleic acid-binding domain in a transcriptional regulator as the full-length protein. Alternatively, the functional domain corresponds to, for example, a kinase domain in a kinase functioning as an intracellular signal factor.

The “encoding nucleic acid” refers to a nucleic acid encoding any of the four polypeptides, i.e., BIL5, BNX1, BIL4, and BIL6, or the variant polypeptide thereof having plant disease resistance-enhancing activity, or the fragment of the polypeptide or the variant polypeptide having the activity. Examples thereof include: a nucleic acid consisting of the nucleotide sequence represented by SEQ ID NO: 3 as a nucleic acid encoding the Arabidopsis thaliana BIL5 amino acid sequence represented by SEQ ID NO: 1; a nucleic acid consisting of the nucleotide sequence represented by SEQ ID NO: 4 as a nucleic acid encoding the Arabidopsis thaliana BNX1 amino acid sequence represented by SEQ ID NO: 2; a nucleic acid consisting of the nucleotide sequence represented by SEQ ID NO: 13 as a nucleic acid encoding the Arabidopsis thaliana BIL4 amino acid sequence represented by SEQ ID NO: 11; and a nucleic acid consisting of the nucleotide sequence represented by SEQ ID NO: 14 as a nucleic acid encoding the Arabidopsis thaliana BIL6 amino acid sequence represented by SEQ ID NO: 12.

In the present invention, the “nucleic acid” mainly refers to a natural nucleic acid such as DNA and/or RNA and can also include artificially chemically modified or constructed nucleic acids or nucleic acid analogs. If necessary, the nucleic acid may be labeled, at its phosphate group, sugar moiety, and/or base moiety, with a nucleic acid-labeling material.

The “wild-type gene” refers to a gene that encodes the wild-type protein, i.e., a protein having its original functions, and typically has a nucleotide sequence most commonly found in the natural world among allele populations of the same type.

The “nucleic acid encoding the variant polypeptide” refers to a nucleic acid encoding the variant polypeptide of BIL5, BNX1, BIL4, or BIL6. This nucleic acid encoding the variant polypeptide includes, for example, a nucleic acid that has a nucleotide sequence derived from the nucleotide sequence of each wild-type gene by the deletion, substitution, or addition of one or several nucleotides, has 40% or more , 50% or more, or 60% or more, preferably 70%, 75%, 80%, or 85%, more preferably 90% or more, 95% or more, 98% or more, or 99% or more identity to the nucleotide sequence of each wild-type gene, or hybridizes under stringent conditions to a nucleic acid fragment comprising a nucleotide sequence complementary to the partial nucleotide sequence of the wild-type gene, and encodes a polypeptide that maintains plant disease resistance-enhancing activity. In this context, the “identity” refers to, between two nucleotide sequences aligned with or without gaps, the ratio (%) of the number of the same bases in one nucleotide sequence to the total number of bases in the other nucleotide sequence. The “several nucleotides” refer to 2 to 60, 2 to 45, 2 to 30, 2 to 14, or 2 to 10, for example, 2 to 8, 2 to 6, 2 to 5, 2 to 4, or 2 or 3 nucleotides. The “stringent conditions” mean conditions under which a nonspecific hybrid is not formed. Examples thereof typically include conditions of low stringency to high stringency. High-stringency conditions are preferred. The low-stringency conditions involve washing, for example, at 42° C. using 5×SSC and 0.1% SDS, preferably washing at 50° C. using 5×SSC and 0.1% SDS, after hybridization. The high-stringency conditions involve washing, for example, at 65° C. using 0.1×SSC and 0.1% SDS, after hybridization. The hybridization conditions are also described in, for example, Sambrook, J. et al., (1989) Molecular Cloning: a Laboratory Manual Second Ed., Cold Spring Harbor Laboratory Press, Cold Spring Harbor, N.Y. Specific examples of such a nucleic acid encoding the variant polypeptide include variants based on a polymorphism such as SNP (single nucleotide polymorphism), splice variants, and variants based on the degeneracy of a genetic code.

The “nucleic acid encoding the fragment thereof” refers to a nucleic acid encoding the fragment of the BIL5, BNX1, BIL4, or BIL6 described above, or the variant polypeptide thereof having plant disease resistance-enhancing activity, wherein the fragment maintains plant disease resistance-enhancing activity.

The “nucleic acid expression system” refers to one expression system unit that can cause expression of the nucleic acid (mainly, a gene or a fragment thereof) incorporated in the system. The nucleic acid expression system has expression regulatory regions essential for gene expression, in addition to the nucleic acid region. The essential expression regulatory regions include, for example, a promoter and a terminator. The system may additionally comprise an enhancer, a poly adenylation signal, a 5′-UTR (untranslated region) sequence, a marker or selective marker gene, a multicloning site, a replication origin, and the like. The nucleic acid expression system includes the whole of one expression system unit necessary for the expression of the particular gene or the like isolated from the genome as well as a system artificially constructed, for example, by combining expression regulatory regions or the like derived from various organisms. Any of these nucleic acid expression systems can be used in the present invention.

In the present specification, the “nucleic acid expression system” provides a plant of interest with the wild-type BIL5, BNX1, BIL4, or BIL6, or the variant polypeptide thereof having plant disease resistance-enhancing activity, or the fragment of the polypeptide or the variant polypeptide having the activity (hereinafter, collectively referred to as a “plant disease resistance polypeptide, etc.”) and has the function of enhancing the disease resistance of the plant.

General plants, however, also usually have an endogenous wild-type BIL5 gene, wild-type BNX1 gene, wild-type BIL4 gene, or wild-type BIL6 gene and have, by nature, disease resistance attributed to the expression of the gene. Hence, in order to allow the disease-resistant plant prepared by the method of the present invention to have more enhanced disease resistance compared with usual plants of the same type thereas, the nucleic acid expression system is required to have the function of increasing the expression level of BIL5, BNX1, BIL4, or BIL6 to more than the usual level in the plant of interest.

Thus, the nucleic acid expression system of the present invention introduced into the plant of interest desirably has properties that allow overexpression of the incorporated nucleic acid encoding the plant disease resistance polypeptide, etc. (hereinafter, referred to as a “plant disease resistance nucleic acid, etc.”), and/or constitutive expression of the incorporated plant disease resistance nucleic acid, etc. or inducible expression of the incorporated plant disease resistance nucleic acid, etc. This exogenous nucleic acid expression system may further have properties that allow maintaining a plurality of its own copies (multicopy) in the plant cell.

The nucleic acid expression system capable of overexpressing the plant disease resistance nucleic acid, etc. for use in the method of the present invention expresses the plant disease resistance nucleic acid, etc. at 2 or more times, preferably 5 or more times, more preferably 10 or more times or 20 or more times, the usual expression level thereof per nucleic acid expression system. Such a nucleic acid expression system is effective because the system can confer disease resistance more than the usual one to the plant by increasing the absolute amount of the plant disease resistance polypeptide, etc. per cell.

The nucleic acid expression system capable of constitutively expressing the plant disease resistance nucleic acid, etc. for use in the method of the present invention can continuously express the plant disease resistance nucleic acid, etc. all the time, regardless of timing or an expression site. Hence, the nucleic acid expression system having this property is very effective because the system can provide the plant disease resistance polypeptide, etc. independently of the temporal or positional control, if any, of the expression level of the endogenous BIL5 gene, BNX1 gene, BIL4 gene, or BIL6 gene.

The nucleic acid expression system capable of inducing the expression of the plant disease resistance nucleic acid, etc. for use in the method of the present invention can express the incorporated plant disease resistance nucleic acid, etc. in a time- or site-specific manner. Thus, the nucleic acid expression system having this property is very effective because the system can provide the plant disease resistance polypeptide, etc. by expressing the plant disease resistance nucleic acid, etc. at an arbitrary time or site independently of the temporal or site-specific control, if any, of the expression of the endogenous BIL5 gene, BNX1 gene, BIL4 gene, or BIL6 gene.

The multicopy nucleic acid expression system for use in the method of the present invention has the advantage that, even if each individual nucleic acid expression system expresses the plant disease resistance nucleic acid, etc. at a low expression level, the nucleic acid expression system itself can increase in number, resulting in an increased expression level per cell as a whole. In the present invention, the multicopy nucleic acid expression system can be used in combination with the overexpressing nucleic acid expression system, the constitutive expression-type nucleic acid expression system, or the inducible expression-type nucleic acid expression system to thereby more effectively confer pathogen resistance to the plant.

The constitution of the exogenous nucleic acid expression system having the property is not particularly limited as long as the system has components necessary for expression and incorporates the plant disease resistance nucleic acid, etc. in an expressible state. In this context, the phrase “incorporating in an expressible state” means that the plant disease resistance nucleic acid, etc. is expressibly inserted in the nucleic acid expression system. Specifically, the plant disease resistance nucleic acid, etc. is placed under the control of a promoter and a terminator in the nucleic acid expression system, i.e., the plant disease resistance nucleic acid, etc. is operably linked thereto. Specific examples of the nucleic acid expression system thus constituted include expression vectors.

In the present invention, the “expression vector” refers to a nucleic acid expression system that can transport the incorporated plant disease resistance nucleic acid, etc. into the plant cell of interest so that the plant disease resistance nucleic acid, etc. can be expressed in the plant cell. Specific examples thereof include expression vectors based on plasmids or viruses.

In the case of the expression vector based on a plasmid (hereinafter, referred to as a “plasmid expression vector”), for example, a vector of pPZP, pSMA, pUC, pBR, pBluescript (Stratagene Corp.), or pTriEX™ (Takara Bio Inc.) series, or a binary vector of pBI, pRI, or pGW series can be used as a plasmid moiety.

In the case of the expression vector based on a virus (hereinafter, referred to as a “viral expression vector”), cauliflower mosaic virus (CaMV), bean golden mosaic virus (BGMV), tobacco mosaic virus (TMV), or the like can be used as a virus moiety.

The expression vector comprises, as described above, a promoter and a terminator as expression regulatory regions. The expression vector may additionally comprise an enhancer, a poly adenylation signal, a 5′-UTR (untranslated region) sequence, a marker or selective marker gene, a multicloning site, a replication origin, and the like. The respective types of these components are not particularly limited as long as these components can exert their functions in the plant cell. Those known in the art can be selected appropriately according to the plant to be transformed or according to purposes (e.g., expression pattern) in the plant.

The promoter used can be selected, for example, according to the desired expression pattern, from an overexpressing promoter, a constitutive promoter, a site-specific promoter, a stage-specific promoter, and/or an inducible promoter. Specific examples of the overexpressing constitutive promoter include cauliflower mosaic virus (CaMV)-derived 35S promoter, Ti plasmid-derived nopaline synthase gene promoter Pnos, maize-derived ubiquitin promoter, Oryza sativa-derived actin promoter, and tobacco-derived PR protein promoter. Ribulose bisphosphate carboxylase small subunit (Rubisco ssu) promoters or histone promoters of various plant species may be used. Specific examples of the site-specific promoter include root-specific promoter described in JP Patent Publication (Kokai) No. 2007-77677 A (2007).

Examples of the terminator include nopaline synthase (NOS) gene terminator, octopine synthase (OCS) gene terminator, CaMV 35S terminator, E. coli lipopolyprotein lpp 3′ terminator, trp operon terminator, amyB terminator, and ADH1 gene terminator. The terminator is not particularly limited as long as its sequence can terminate the transcription of the gene transcribed by the action of the promoter.

Examples of the enhancer include an enhancer region containing an upstream sequence in the CaMV 35S promoter. The enhancer is not particularly limited as long as the enhancer can enhance the expression efficiency of the plant disease resistance nucleic acid, etc.

Examples of the marker or selective marker gene include drug resistance genes (e.g., tetracycline resistance gene, ampicillin resistance gene, kanamycin resistance gene, hygromycin resistance gene, spectinomycin resistance gene, chloramphenicol resistance gene, and neomycin resistance gene), fluorescent or luminescent reporter genes (e.g., luciferase, β-galactosidase, β-glucuronidase (GUS), and green fluorescence protein (GFP) genes), and enzyme genes such as neomycin phosphotransferase II (NPT II), dihydrofolate reductase, and Blasticidin S resistance genes. The marker or selective marker gene may be ligated with the same expression vector as the expression vector incorporating the plant disease resistance nucleic acid, etc. or may be ligated with an expression vector different therefrom. In the latter case, both the expression vectors can be cointroduced into the plant of interest to thereby produce effects equivalent to those brought about by introduction of the single expression vector incorporating the plant disease resistance nucleic acid, etc. and the gene.

### (2) Preparation of Nucleic Acid Expression System

In the method of the present invention, the nucleic acid expression system to be introduced into the plant of interest can be prepared according to a method known in the art, for example, a method described in Sambrook, J. et al., (1989) Molecular Cloning: a Laboratory Manual Second Ed., Cold Spring Harbor Laboratory Press, Cold Spring Harbor, N.Y.

Hereinafter, the preparation of the plasmid expression vector or the viral expression vector will be described with reference to specific examples, though the preparation of the nucleic acid expression system is not limited thereto.

### (2-1) Preparation of Plasmid Expression Vector

First, of the plant disease resistance nucleic acid, etc. described above, the desired nucleic acid is cloned. For example, in the case of cloning the Arabidopsis thaliana BIL5 gene, an appropriate region is selected from the nucleotide sequence represented by SEQ ID NO: 3, and an oligonucleotide having the nucleotide sequence thereof is chemically synthesized. The chemical synthesis can utilize a custom synthesis service provided by each life science manufacturer.

Next, the BIL5 gene is isolated with the oligonucleotide as a probe from an Arabidopsis thaliana cDNA library according to a method known in the art. For the details of the isolation method, see Sambrook, J. et al., (1989) (supra). Since the Arabidopsis thaliana cDNA library is commercially available from each life science manufacturer such as Stratagene Corp., such a commercially available product may be used. Alternatively, oligonucleotides serving as a primer pair may be chemically synthesized on the basis of the nucleotide sequence represented by SEQ ID NO: 3, and the BIL5 gene of interest can be amplified from an Arabidopsis thaliana genomic DNA or cDNA library by a nucleic acid amplification method such as PCR using the primer pair. In the case of performing the nucleic acid amplification, high-fidelity DNA polymerase having 3′-5′ exonuclease activity, such as Pfu polymerase, is preferably used. For detailed conditions, etc., for the nucleic acid amplification, see, for example, a method described in Innis M. et al (Ed.), (1990), Academic Press, PCR Protocols: A Guide to Methods and Applications. The isolated BIL5 gene is inserted, if necessary, to an appropriate plasmid and cloned in a host microbe such as E. coli. Then, its full-length nucleotide sequence is determined according to a publicly known technique.

Subsequently, the BIL5 gene is integrated into a predetermined site in the backbone portion of the desired nucleic acid expression system. For example, the BIL5 gene is cleaved with appropriate restriction enzymes on the basis of the determined nucleotide sequence. Meanwhile, the nucleic acid expression system is cleaved at the corresponding restriction enzyme sites. A multicloning site, if present, in the nucleic acid expression system is conveniently used. Subsequently, the BIL5 gene is inserted to the nucleic acid expression system by the ligation of both the nucleic acids at their ends using ligase or the like to complete the nucleic acid expression system for the BIL5 gene of interest. These series of gene manipulation procedures are technically well known in the art. For the details of the method, see, for example, Sambrook, J. et al., (1989) (supra).

### (2-2) Preparation of Viral Expression Vector

Basic procedures therefor can follow the method for preparing the plasmid expression vector. First, a plant virus genome is prepared by a method known in the art and then inserted to an appropriate cloning vector, for example, of E. coli-derived pBI, pPZP, pSMA, pUC, pBR, or pBluescript series, to obtain a recombinant. Next, the plant disease resistance nucleic acid, etc. is inserted to a predetermined site in the viral genome contained in the recombinant, and cloned. Subsequently, a plant virus genome region can be excised from the recombinant using restriction enzymes. In this way, the viral expression vector of interest can be obtained.

(3) Method for Introducing Nucleic Acid Expression System into Plant Cell

A method for introducing the nucleic acid expression system incorporating the plant disease resistance nucleic acid, etc. into a plant cell, i.e., a method for transforming a plant cell may be any appropriate method known in the art. In the case of using a plasmid expression vector as the nucleic acid expression system, for example, a protoplast, particle gun, or Agrobacterium method can be used preferably as the transformation method.

The protoplast method involves removing cell walls from plant cells by enzymatic (e.g., cellulase) treatment and transferring the gene of interest into the resulting plant cells (protoplasts). This method can be further classified into electroporation, microinjection, and polyethylene glycol methods, etc., depending on an approach for the gene transfer. The electroporation method involves applying an electric pulse to a mixed solution of the protoplast and the gene of interest to transfer the gene into the protoplast. The microinjection method involves directly transferring the gene of interest into the protoplast under a microscope using a microneedle. The polyethylene glycol method involves transferring the gene of interest to the protoplast by the action of polyethylene glycol.

The particle gun method involves attaching the gene of interest to microparticles of gold, tungsten, or the like, and intracellularly injecting the resulting microparticles into plant tissues cells using high-pressure gas to introduce the gene of interest into the cells. This method can produce transformed cells containing the gene of interest integrated in the genomic DNA of the host plant cells. The transformed cells are usually screened on the basis of the marker gene product in the nucleic acid expression system.

The Agrobacterium method involves transforming plant cells using, as transforming factors, a bacterium of the genus Agrobacterium (e.g., A. tumefaciens land A. rhizogenes) and Ti plasmid, Ri plasmid, or the like derived therefrom. This method can introduce the gene of interest into the genomic DNA of host plant cells.

These methods are all known in the art. For the details thereof, see appropriate protocols described in, for example, Shokubutsu Taisha Kogaku (Plant Metabolic Engineering in English) Handbook (2002, NTS Inc.) or Shinban Model Shokubutsu No Jikken Protocol: Idengakuteki Shuhou Kara Genome Kaiseki Made (Experimental Protocol of Model Plant, New Edition: From Genetic Technique To Genomic Analysis in English) (2001, Shujunsha Co., Ltd.).

Alternatively, in the case of using a viral expression vector (e.g., using CaMV, BGMV, or TMV described above) as the nucleic acid expression system, the plant cells of interest can be infected with the viral expression vector containing the integrated plant disease resistance nucleic acid, etc. to obtain transformed cells. For the details of such a gene introduction method using the viral vector, see, for example, the method of Hohn et al. (Molecular Biology of Plant Tumors (Academic Press, New York) 1982, pp. 549) and U.S. Pat. No. 4,407,956.

The present invention does not require that the plant species from which the plant disease resistance nucleic acid, etc. is derived should be identical to the plant species of plant cells to be transformed. For example, the nucleic acid expression system incorporating the BIL5 gene derived from Arabidopsis thaliana of the family Brassicaceae may be introduced into the cells of tobacco (Nicotiana tabacum) of the family Solanaceae. This is because: the BR signaling pathway universally exists in plants; and individual signaling factors are highly conservative among species; thus, even if a nucleic acid expression system derived from a different species is introduced, the ortholog of the plant disease resistance nucleic acid, etc. inserted in the nucleic acid expression system can exert functions in the species of transformed plant similar to those brought about by introduction of the system of the same plant species.

In the present invention, preferably, the plant into which the nucleic acid expression system is introduced has at least a wild-type gene cluster involved in the BR signaling pathway and a wild-type gene cluster involved in the natural immunity-inducing signaling pathway mediated by salicylic acid. This is because: the enhancement in plant disease resistance of the present invention is based on the enhanced signals of these pathways; and even if signals are enhanced by increasing the expression level of a polypeptide located upstream in the signaling pathway, subsequent signals cannot be transduced in the presence of its downstream factor having a loss-of-function mutation or the like, resulting in unsuccessful obtainment of the disease-resistant plant.

In the present invention, two or more nucleic acid expression systems differing in the plant disease resistance nucleic acid, etc. incorporated therein can be introduced, in a range that can coexist with each other, into one plant cell. For example, a nucleic acid expression system incorporating the Oryza sativa BIL5 gene and a nucleic acid expression system incorporating the Oryza sativa BNX1 gene may be introduced into one plant cell (e.g., Oryza sativa cell), or nucleic acid expression systems incorporating the Oryza sativa BIL5 gene, the Oryza sativa BIL4 gene, and the Oryza sativa BIL6 gene, respectively, may be introduced thereinto.

After this step, the transformed plant cell can be regenerated into a transgenic plant according to a publicly known method. Examples thereof include an in vitro regeneration method which involves regenerating plant bodies through the formation of callus composed of undifferentiated grown cells. This method is known in the art. For the details thereof, see, for example, Shokubutsu Taisha Kogaku (Plant Metabolic Engineering in English) Handbook (2002, NTS Inc.) or Shinban Model Shokubutsu No Jikken Protocol: Idengakuteki Shuhou Kara Genome Kaiseki Made (Experimental Protocol of Model Plant, New Edition: From Genetic Technique To Genomic Analysis in English) (2001, Shujunsha Co., Ltd.) described above. Alternatively, an in planta method may be used, which involves directly introducing the nucleic acid expression system to the cells of the plant individual of interest without the callus or cell culture step. A phytohormone such as auxin, gibberellin, and/or cytokinin may be used for promoting the growth and/or division of the transformed cells.

The first-generation transgenic plant thus obtained by the method is a disease-resistant plant. In the present invention, this first-generation transgenic plant also encompasses clones having genetic information identical thereto. The clones correspond to, for example, a plant obtained by the cutting, grafting, or layering of a portion of the plant body collected from the first-generation transgenic plant, a plant body regenerated through callus formation from cultured cells, or a vegetative plant newly formed vegetative reproductive organs (e.g., rhizomes, tuberous roots, corms, and runners) obtained by asexual reproduction from the first-generation transgenic plant.

The plant disease resistance polypeptide is expressed from the nucleic acid expression system introduced into this disease-resistant plant. Its expression level per cell of the plant is increased compared with the wild-type individual of the same type thereas. As a result, the natural immune system is enhanced to improve disease resistance.

### 2. Method for Obtaining Progeny of Disease-Resistant Plant

The second embodiment of the present invention relates to a method for obtaining progeny of the disease-resistant plant. In the present specification, the “progeny of the disease-resistant plant” refers to offspring that is obtained via sexual reproduction from the first-generation transgenic plant obtained by the method of the first embodiment and retains the nucleic acid expression system according to the first embodiment. The progeny corresponds to, for example, a seedling of the first-generation transgenic plant.

The progeny can be obtained from the disease-resistant plant of the present invention by a publicly known method. For example, the disease-resistant plant as the first-generation transgenic plant is allowed to fruit, and seeds can be obtained as first-generation progeny and also as a second-generation transgenic plant. As an example of a method for further obtaining second-generation progeny from the first-generation progeny of the present invention, the seeds are rooted on an appropriate medium, and the resulting shoots are transplanted in a pot containing soil. The second-generation progeny can be obtained by growth under appropriate cultivation conditions. The progeny according to this embodiment is not limited by its generation as long as the progeny retains the nucleic acid expression system according to the first embodiment. Thus, third or later generation progeny can be obtained by repeating a method similar to the method for obtaining the second-generation progeny.

### 3. Disease-Resistant Plant

The third embodiment of the present invention relates to a disease-resistant plant. The disease-resistant plant of the present invention has substantially the same constitution as in the disease-resistant plant obtained by the preparation method of the first embodiment or the progeny obtained by the obtainment method of the second embodiment.

Specifically, the disease-resistant plant according to this embodiment encompasses all transgenic plants, regardless of generations, as long as the transgenic plants each comprise at least one exogenous nucleic acid expression system incorporating, in an expressible state, a nucleic acid encoding any of the four plant disease resistance polypeptides, i.e., the wild-type BIL5, BNX1, BIL4, and BIL6, or the variant polypeptide thereof having plant disease resistance-enhancing activity, or the fragment of the polypeptide or the variant polypeptide having the activity.

The “exogenous nucleic acid expression system” refers to a foreign nucleic acid expression system introduced from outside via artificial operation. Thus, the exogenous nucleic acid expression system does not correspond to an endogenous nucleic acid expression system originally located at a predetermined locus on the plant genome. However, even such an endogenous nucleic acid expression system, when mutated from outside via artificial operation such as mutagenesis or whose origin is derived from an exogenous nucleic acid expression system, as in the progeny of the transgenic plant, is also included in the exogenous nucleic acid expression system of the present invention.

Each constitution of the disease-resistant plant is as described in the first embodiment, so that the detailed description thereof is omitted here.

### 4. Plant Disease Resistance-Enhancing Agent

The fourth embodiment of the present invention relates to a plant disease resistance-enhancing agent. The plant disease resistance-enhancing agent of the present invention comprises at least one of wild-type BIL5, wild-type BNX1, wild-type BIL4, and wild-type BIL6, or variant polypeptides thereof having plant disease resistance-enhancing activity, or polypeptide fragments thereof having plant disease resistance-enhancing activity, as an active ingredient. As described above, the wild-type BIL5, the wild-type BNX1, the wild-type BIL4, or the wild-type BIL6 functions as an intracellular signaling factor in the BR signaling pathway. Thus, any of these wild-type polypeptides, or the variant polypeptide thereof having plant disease resistance-enhancing activity, or the fragment of the polypeptide or the variant polypeptide having the activity can be applied to the plant of interest and allowed to function intracellularly to thereby enhance the plant disease resistance of the plant of interest.

The amount of the wild-type BIL5, the wild-type BNX1, the wild-type BIL4, or the wild-type BIL6, or the variant polypeptide thereof, or the fragment of the polypeptide or the variant polypeptide contained in the plant disease resistance-enhancing agent of the present invention differs depending on various conditions such as the type of the polypeptide contained therein, the type of a carrier contained therein, the type of a recipient plant, the purpose of application, an application method, and the type of an additional drug, if contained, having pharmacological effects. The content thereof can be determined in consideration of conditions capable of conferring disease resistance to the target plant after application of the plant disease resistance-enhancing agent.

The plant disease resistance-enhancing agent of the present invention may also comprise, if necessary, an agriculturally acceptable carrier. The “agriculturally acceptable carrier” refers to a substance that facilitates the application of the plant disease resistance-enhancing agent and inhibits or suppresses the decomposition of the polypeptide serving as an active ingredient or/and controls the rate at which the polypeptide acts. Preferably, the agriculturally acceptable carrier has no harmful effect or only small influence on environments such as soil and water quality or on animals, particularly, humans, when applied to the plant of interest.

Examples of the carrier include natural mineral powders, synthetic mineral powders, emulsifiers, dispersants, and surfactants.

Examples of the natural mineral powders include kaolin, clay, talc, and chalk.

Examples of the synthetic mineral powders include highly dispersible silica and silicate. Examples of the emulsifiers include nonionic emulsifiers and anionic emulsifiers (e.g., polyoxyethylene fatty alcohol ether, alkyl sulfonate, and aryl sulfonate).

Examples of the dispersants include lignosulfite waste liquors and methylcellulose.

Examples of the surfactants include alkali metals salts, alkaline earth metal salts, and ammonium salts of lignosulfonic acid, naphthalenesulfonic acid, phenolsulfonic acid, and dibutylnaphthalenesulfonic acid, alkylaryl sulfonate, alkyl sulfate, alkyl sulfonate, fatty alcohol sulfate, fatty acid and sulfated fatty alcohol glycol ethers, condensates of sulfonated naphthalene or a naphthalene derivative and formaldehyde, condensates of naphthalene or naphthalenesulfonic acid, phenol, and formaldehyde, polyoxyethylene octyl phenyl ether, ethoxylated isooctylphenol, octylphenol, nonylphenol, alkylphenyl polyglycol ether, tributylphenyl polyglycol ether, tristearylphenyl polyglycol ether, alkylaryl polyether alcohol, condensates of alcohol or fatty alcohol and ethylene oxide, ethoxylated castor oil, polyoxyethylene alkyl ether, ethoxylated polyoxypropylene, lauryl alcohol polyglycol ether acetal, sorbitol ester, lignosulfite waste liquors, and methylcellulose.

The amount of the BIL5, BNX1, BIL4, or BIL6 described above, or the variant polypeptide thereof, or the fragment of the polypeptide or the variant polypeptide contained in the plant disease resistance-enhancing agent of the present invention differs depending on various conditions such as the type of the plant branching inhibitor contained therein, the type of a carrier contained therein, the type of a recipient plant, the purpose of application, an application method, and the type of an additional drug, if contained, having pharmacological effects. The content of the plant branching inhibitor in the plant branching-inhibiting composition can be determined within the scope of the technical common sense in the art in consideration of conditions under which the plant branching inhibitor contained in the composition achieves the desired amount for the target plant after application.

### 5. Method for Enhancing Plant Disease Resistance

The fifth embodiment of the present invention relates to a method for enhancing the plant disease resistance of a plant of interest, comprising applying the plant disease resistance-enhancing agent according to the fourth embodiment to the plant.

The form in which the plant disease resistance-enhancing agent according to the fourth embodiment is applied thereto can be set to a routine pharmaceutical form, for example, solutions, oily dispersions, emulsions, suspensions, dusts, powders, pastes, pellets, tablets, and granules, which permit direct spraying, coating, and/or dipping.

The method for applying the plant disease resistance-enhancing agent according to the fourth embodiment is not particularly limited as long as the application method can enhance the disease resistance of the plant of interest. The plant disease resistance-enhancing agent can be applied by a method known in the art.

For example, for hydroponic cultivation, the plant disease resistance-enhancing agent can be added into a hydroponic solution. The applied plant disease resistance-enhancing agent is absorbed from the root and thereby spread throughout the plant where the active ingredient can then exert the effects of the present invention.

Alternatively, the plant disease resistance-enhancing agent may be applied directly or indirectly to soil. In this case, note that the polypeptide serving as the active ingredient of the present invention tends to be decomposed in a relatively short time by the action of microbes or the like in soil. Thus, the polypeptide may be enclosed in a sustained-release inclusion body and applied indirectly to the plant of interest. In this case as well, the applied plant disease resistance-enhancing agent of the present invention is absorbed from the root and thereby spread throughout the plant where the active ingredient can then exert the effects of the present invention in each cell.

In addition, a solution containing the plant disease resistance-enhancing agent dissolved therein may be applied to the plant body by coating, dipping, injection, or spraying. The site of the coating or the like is not limited and can be any desired position such as a stem or the base of the petiole.

The amount of the plant disease resistance-enhancing agent of the present invention applied varies depending on the type of the plant disease resistance peptide, etc., contained in the plant disease resistance-enhancing agent used, or the type of a recipient plant. The amount of the plant disease resistance-enhancing agent applied even to target plants of the same type varies between hydroponic cultivation and soil cultivation. This is because the plant disease resistance-enhancing agent of the present invention is generally decomposed by the action of microbial decomposition in soil at a faster rate than that in a hydroponic solution. Thus, the amount of the plant disease resistance-enhancing agent applied can be determined appropriately according to situations, purposes, and needs by those skilled in the art.

### EXAMPLES

### Example 1

<Induction of PR1 Gene Expression in Gain-of-Function bil5 Mutant>

The gene expression of a pathogen resistance marker PR1 (pathogenesis related 1) was tested in bil5-1D, a gain-of-function BIL5 mutant of Arabidopsis thaliana. PR1 is an antibacterial protein whose expression is generally induced by the bacterial pathogen infection of a plant, and is known to typically function downstream in the pathogen resistance signaling pathway mediated by salicylic acid. Thus, the increased expression of the PR1 gene in the bacterial pathogen-uninfected bil5-1D strain suggests that this bil5-1D strain has acquired disease resistance.

### (Method)

First, grown Arabidopsis thaliana individuals of wild-type and bil5-1D strains were separately used in RNA extraction 10 days after seeding to soil. For this extraction, BR-unsupplemented (untreated) and BR-supplemented (treated) samples were prepared for each strain. According to the report of Nakashita et al. (The Plant Jour., 2003, 33: 887-898), BR increases the expression of the PR1 gene when administered to Arabidopsis thaliana. Total RNA was extracted from the rosette leaf of each strain using RNeasy Plant Mini Kit (Qiagen N.V.). Next, cDNA was synthesized from the extracted total RNA using Prime Script First-Strand cDNA Synthesis Kit (Takara Bio Inc.). Specific procedures for the total RNA extraction and the cDNA synthesis followed the protocol included in each kit. Subsequently, the expression of the PR1 gene was analyzed by real-time PCR with the synthesized cDNA as a template using primers RT-PR1-F consisting of the nucleotide sequence represented by SEQ ID NO: 5 and PR1-R consisting of the nucleotide sequence represented by SEQ ID NO: 6, SYBR Premix EX Taq kit (Takara Bio Inc.) and a real-time PCR apparatus Thermal Cycler Dice (Takara Bio Inc.).

### (Results)

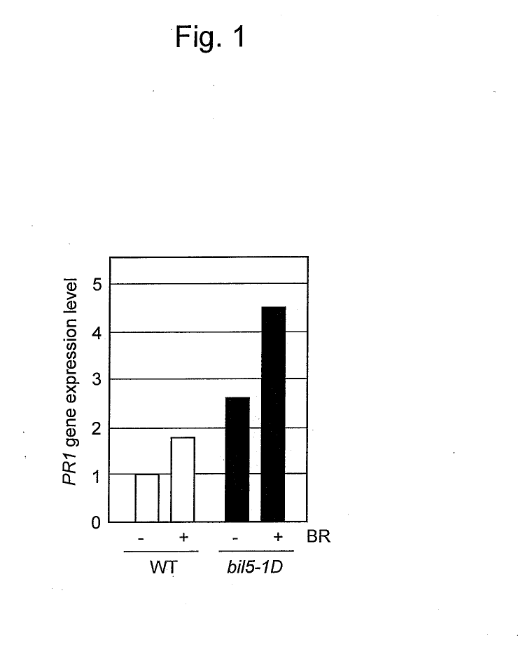

The results are shown in FIG. 1. In this diagram, the expression level of the PR1 gene is indicated by a relative value with the value of the BR-untreated (−BR) wild-type strain (WT) as 1. As is also evident from FIG. 1, the bil5-1D strain, even in the BR-untreated state, had the higher expression level of the PR1 gene than that of the BR-treated (+BR) wild-type strain. Also, the BR-treated bil5-1D strain exhibited the further enhanced expression level of the PR1 gene. These results suggested that bil5-1D has already acquired disease resistance.

### Example 2

### <Induction of PR1 Gene Expression in Overexpressing Strain>

Example 1 showed that the expression of the PR1 gene is increased in the gain-of-function BIL5 mutant. Thus, whether the overexpression of the BIL5 gene or the like also increased PR1 gene expression was tested. This Example employed a double transgenic strain BIL5-OX1×BNX1-OX1 of a wild-type BIL5 gene-overexpressing transgenic strain BIL5-OX1 of Arabidopsis thaliana and a wild-type BNX1 gene-overexpressing transgenic strain BNX1-OX1 of Arabidopsis thaliana. This is because a strain that was found most morphologically similar to the bil5-1D mutant in morphological observation was also used in the analysis of the PR1 gene. This is also because strains overexpressing the wild-type strain-derived BIL5 gene alone were morphologically similar to the bil5-1D mutant in terms of the forms of hypocotyl and matured leaves or stems, but merely exhibited a mild abnormal character, whereas the strong abnormal character exhibited by the bil5-1D mutant was reproduced only by preparing the double transgenic strain BIL5-OX1×BNX1-OX1 of the wild-type BIL5 gene-overexpressing transgenic strain BIL5-OX1 strain of Arabidopsis thaliana and the wild-type BNX1 gene-overexpressing transgenic strain BNX1-OX1 strain of Arabidopsis thaliana. This is further because the bil5-1D mutant was shown to also highly express the BNX1 gene adjacent to the BIL5 gene under the principle that a mutation demethylating one site on the BIL5 gene presumably functions as a gene expression promoter for its adjacent BNX1 gene.

### (Method)

### (1) Cloning of Wild-Type BIL5 and BNX1 Genes

(Preparation of Total RNA and Synthesis of cDNA)

RNeasy Plant Mini Kit (Qiagen N.V.) was used in total RNA extraction. First, less than 0.1 mg (fresh weight) of the rosette leaf was collected, then frozen in liquid nitrogen, and then disrupted using a mortar. To the disrupted sample, 450 μL of a β-mercaptoethanol (10 μL)/buffer RLT (1 μL) mixed solution was added, and the mixture was vortexed. Specific procedures therefor followed the protocol included in the kit. Finally, total RNA obtained by ethanol precipitation was dissolved in 50 μL of RNase-free water.

Super Script III First-Strand Synthesis System for RT-PCR (Invitrogen Corp.) was used in cDNA synthesis. Specific procedures therefor followed the protocol included in the kit.

(Preparation of Cloning Vector)

In order to obtain the full-length ORFs of the BIL5 gene and the BNX1 gene, PCR reaction was performed using the prepared cDNA libraries and KOD-plus-DNA polymerase (Toyobo Co., Ltd.). Primers consisting of the nucleotide sequences represented by SEQ ID NOs: 7 and 8 and SEQ ID NOs: 9 and 10 were used as BIL5 and BNX1 forward and revers primers, respectively.

The BIL5 gene and the BNX1 gene were cloned using pENTR/D TOPO cloning kit (Invitrogen Corp.). Specific procedures therefor followed the protocol included in the kit. In this way, a BIL5 gene cloning vector pENTR-BIL5 and a BNX1 gene cloning vector pENTR-BNX1 were obtained.

(Preparation of Expression Vector)

Expression vectors for plant transformation were produced through LR reaction according to the Gateway technique using pGWB5 vectors (Nakagawa et al., 2007, JBB, 104: 34-41). A mixed solution of pENTR-BIL5 or pENTR-BNX1, the pGWB5 vector, and LR Clonase was prepared and left standing at 25° C. for 1 hour. Then, 1 μL of protease K was added thereto, and the mixture was left standing at 37° C. for 10 minutes. After subsequent incubation at 85° C. for 10 minutes, the reaction solution was mixed with DH5α competent cells, which were then applied to an LB medium containing 25 μg/mL each of kanamycin and hygromycin and cultured overnight. Plasmids were extracted from the resulting transformants to obtain the pGWB5-BIL5 and pGWB5-BNX1 vectors of interest.

(2) Production of Transgenic Arabidopsis thaliana

(Introduction of pGWB5-BIL5 or pGWB5-BNX1 into Agrobacterium)

2 μL of pGWB5-BIL5 or pGWB5-BNX1 with respect to 50 μL of Agrobacterium competent cells was added thereto, then well mixed, and left standing for 30 minutes in ice. Subsequently, the cells were left standing for 1 minute in liquid nitrogen and then thawed in a block incubator set to 37° C. After addition of 250 μL of a YEP medium, the cells were cultured for 60 minutes with shaking at 200 rpm. The cells were inoculated to a YEP medium containing 25 μg/mL each of kanamycin and hygromycin and 50 μg/mL rifampicin and cultured over two nights. The successful introduction of the vectors was confirmed by colony PCR.

(Agrobacterium Infection of Plant)

The colony thus transformed with each vector was precultured overnight in a YEP liquid medium. The volume of the culture solution was brought up to 500 ml, followed by overnight culture. The culture solution was centrifuged at 5000 rpm for 10 minutes, and the supernatant was discarded. The pellet was suspended in an MS medium containing 5% (w/v) sucrose. A pod-removed wild-type Arabidopsis thaliana strain was transformed with pGWB5-BIL5 or pGWB5-BNX1 by the flower dipping method. The obtained T₁ seeds were screened in an MS medium containing 25 μg/mL kanamycin. The obtained transformant was subjected to morphological observation.

### (3) Production of Double Overexpression Strain

The BIL5-overexpressing strain and the BNX1-overexpressing strain were separately analyzed by real-time PCR according to the method of Example 1. The overexpressing strains confirmed to overexpress the BIL5 gene or the BNX1 gene were used. A double overexpression mutant was produced by artificial crossing in which the pollens of one of these overexpressing strains were transferred to the pistil of the other strain. The successful obtainment of the double overexpression strain was confirmed by real-time PCR analysis according to the method of Example 1.

### (4) Induction of PR1 Gene Expression in Double Overexpression Strain

The seeds of wild-type, bil5-1D, and thus-prepared BIL5-OX1×BNX1-OX1 strains of Arabidopsis thaliana were separately sowed and cultivated for 14 days. Specific procedures for total RNA extraction from each strain, cDNA synthesis, and the real-time PCR analysis of PR1 gene expression followed the method of Example 1.

### (Results)

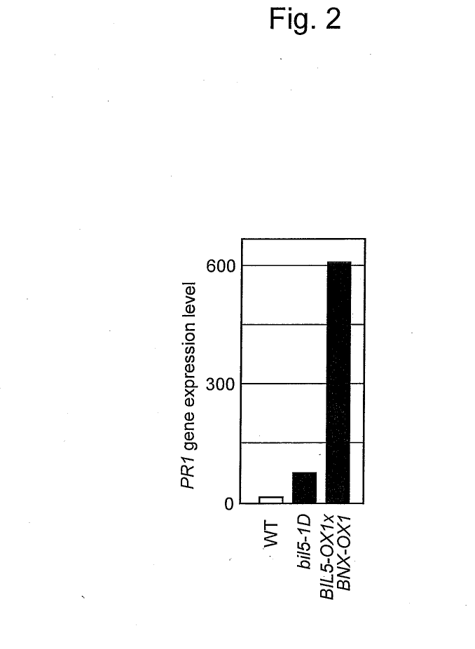

The results are shown in FIG. 2. The much higher expression of the PR1 gene than that in the gain-of-function BIL5 mutant bil5-1D was detected in the BIL5-OX1×BNX1-OX1 strain. Thus, the expression of the PR1 gene was found to be also increased in the BIL5 gene-overexpressing strain, as in bil5-1D. The results also demonstrated that: the BNX1 gene located adjacent to the BIL5 gene on the genome is similar in activity to the BIL5 gene; and the expression of the PR1 gene is remarkably increased in the double mutant thereof. These results suggested that the strains overexpressing the BIL5 and BNX1 genes are capable of conferring disease resistance to the plants.

### Example 3

### <Relationship Between BIR5 and Pathogen Resistance>

In Examples 1 and 2, the increased expression of the PR1 gene was confirmed in the gain-of-function BIL5 mutant and the BIL5-overexpressing strain. Thus, whether these strains actually acquired pathogen resistance was tested.

### (Method)

The successful acquisition of pathogen resistance was confirmed using tobacco bacterial pathogen assay. The plants used were a wild-type Arabidopsis thaliana strain, a bil5-1D strain, a bil5-1D/sid2 double mutant (bil5-1D×sid2), and a sid2 mutant. SID2 is a salicylic acid biosynthetic enzyme. The sid2 mutant, which is deficient in SID2, fails to synthesize salicylic acid and loses disease resistance. First, the seeds of each plant were sowed in soil, and a plant body was then subjected to bacterial inoculation on the 3rd week. A tobacco bacterial pathogen Pseudomonas syringae pv. tabaci (Pst) was cultured at 28° C. for 2 days in a nutrient medium. The bacterial suspension was adjusted to 2×10⁵ CFU (colony forming unit)/ml with 10 mM MgCl₂. Bacterial inoculation was performed by the penetration of the bacterial suspension using a 1-ml needleless syringe. On days 0, 1, 3, and 5 after the infection, the leaves were collected in a disc form from the bacterium-penetrated portions of the leaves. Three discs derived from each plant were combined and homogenized with 10 mM MgCl₂. The CFU count was evaluated after dilution of the homogenate and subsequent growth in a nutrient medium on an agar plate. Three plant bodies derived from each strain were used at each point in time, while two samples were prepared from each plant body.

### (Results)

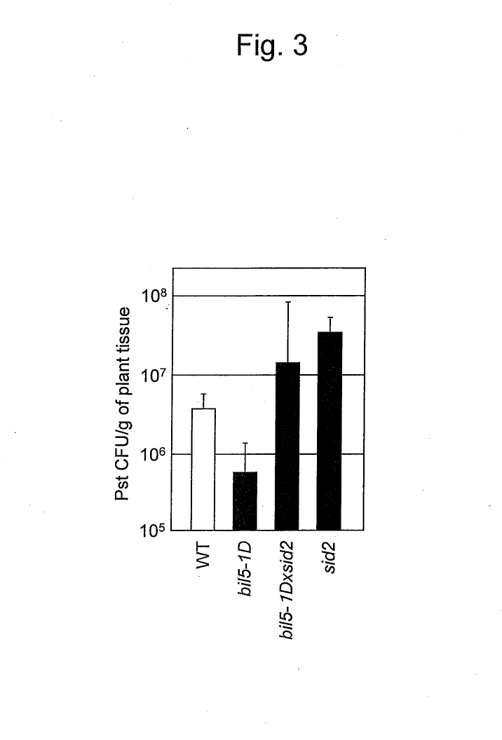

The results are shown in FIG. 3. The ordinate of FIG. 3 denotes the number of colonies of the bacterial pathogen Pst per g of plant tissues. The smaller number of colonies means that the growth of the bacterial pathogen was more suppressed, i.e., disease resistance was acquired. As expected in Examples 1 and 2, the bil5-1D strain was confirmed to acquire disease resistance more than the wild-type strain (WT). By contrast, the bacterial pathogen grew in the bil5-1D/sid2 double mutant (bil5-1D×sid2) at the same level as in the wild-type strain. This indicates that the disease resistance of bil5-1D was suppressed by the inhibition of salicylic acid biosynthesis.

### Example 4

### <Relationship Between BIR5 and Amount of Salicylic Acid Synthesized>

The results of Examples 1 to 3 suggested that pathogen resistance based on the increased expression of the BIL5 gene, i.e., the increased BIL5 level, is acquired via the salicylic acid-mediated disease resistance signaling pathway. Thus, the amount of salicylic acid in a plant body was examined in order to test whether or not the amount of salicylic acid synthesized was increased in the bil5-1D strain.

### (Method)

Leaves were collected together from each grown plant of Example 3 on the 3rd week after seeding to soil, and the level of free salicylic acid (SA) was measured. The SA measurement followed the methods of Nakashita et al. (supra) and Yoshioka et al. (Plant L., 2001, 25, 149-157).

### (Results)

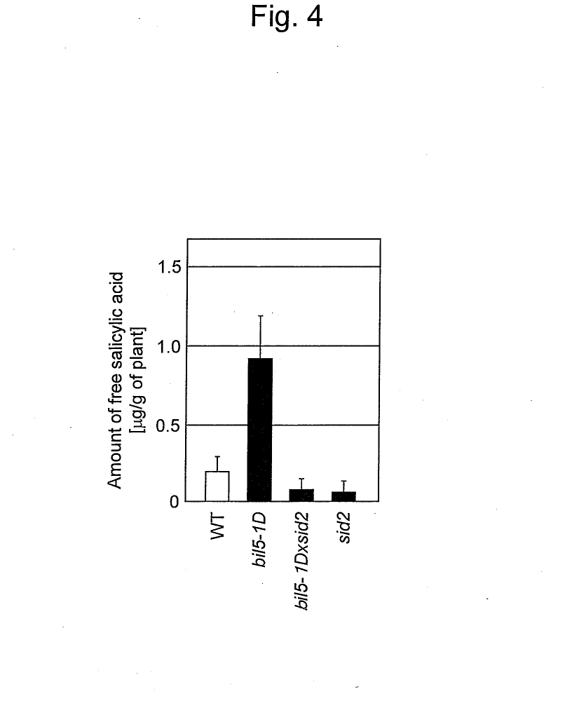

The results are shown in FIG. 4. As expected, free salicylic acid was detected in the bil5-1D strain in 4 or more times the amount of free salicylic acid in the wild-type strain. Thus, the bil5-1D strain was shown to acquire pathogen resistance more than the wild-type strain by virtue of salicylic acid biosynthesized and/or accumulated in an amount larger than the usual amount. By contrast, the amount of salicylic acid detected in the bil5-1D×sid2 double mutant was decreased to the same level as in the mutant having the sid2 mutation alone. These results demonstrated that: the acquisition of disease resistance induced by BIL5 requires salicylic acid; and BIL5 is probably involved, upstream of SID2, in the salicylic acid-mediated disease resistance signaling pathway.

### Example 5

### <Relation of BR Signaling Mutant to Disease Resistance>

The BNX1 gene, which is another gene disclosed in the present invention, was also tested for its involvement in the acquisition of plant pathogen resistance, as in the BIL5 gene.

### (Method)

Wild-type, bil5-1D, BIL5-OX1×BNX1-OX1, and bill strains of Arabidopsis thaliana were used in the experiment.

In this context, the bill strain is a gain-of-function mutant in which BIL1 protein is stabilized and highly accumulated. The BIL1 protein is a downstream factor functioning in the BR intracellular signaling pathway and is known as a transcriptional regulator that controls development. The studies of the present inventors have revealed that the BIL1 protein cannot induce disease resistance.

Basic procedures for pathogen resistance acquisition followed the method of Example 3.

### (Results)

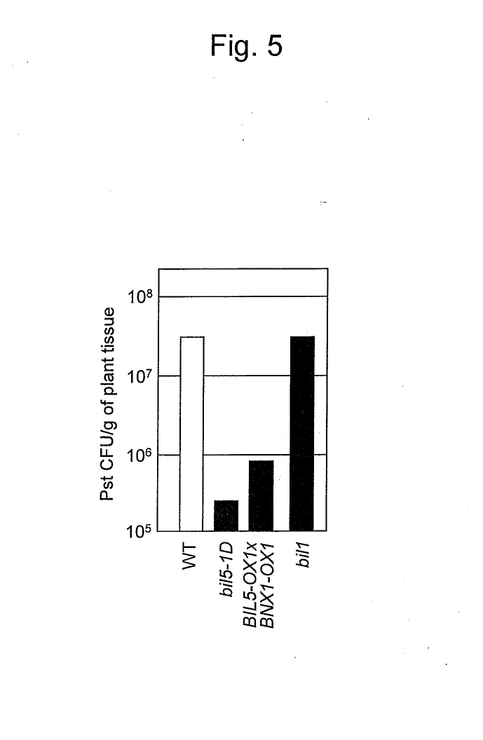

The results are shown in FIG. 5. The bil5-1D and BIL5-OX1×BNX1-OX1 strains were confirmed to have disease resistance more than the wild-type strain (WT). By contrast, the bill strain, which lacks the disease resistance induction mechanism, had disease resistance at the same level as in the wild-type strain. These results demonstrated that the overexpression of the BIL5 gene and the BNX1 gene allows the plant body to acquire disease resistance.

### Example 6

The amount of salicylic acid in each plant body used in Example 5 was examined in order to test whether or not the amount of salicylic acid synthesized was increased in the plant body.

### (Method)

Basic procedures therefor followed the method described in Example 4.

### (Results)

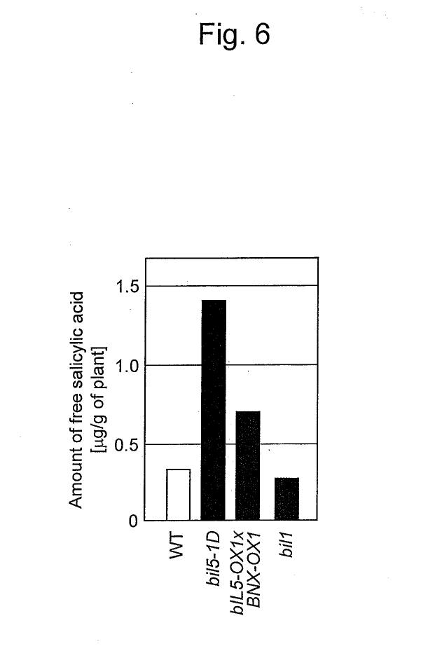

The results are shown in FIG. 6. The amount of salicylic acid larger than that in the wild-type strain (WT) was detected in the bil5-1D and BIL5-OX1×BNX1-OX1 strains, demonstrating that the increased amount of salicylic acid synthesized and/or accumulated is related to the acquisition of disease resistance shown in Example 5 and FIG. 5.

### Example 7

### <Induction of PR1 and PR5 Gene Expression in BIL4 Gene-Overexpressing Strain>

Whether the overexpression of the BIL4 gene increased the expression of pathogen resistance marker PR1 and PR5 genes was tested.

### (Method)

Basic procedures therefor followed the method described in Example 2. This Example employed wild-type, bil5-1D, and wild-type BIL4 gene-overexpressing transgenic BIL4-OX1 strains of Arabidopsis thaliana.

### (Results)

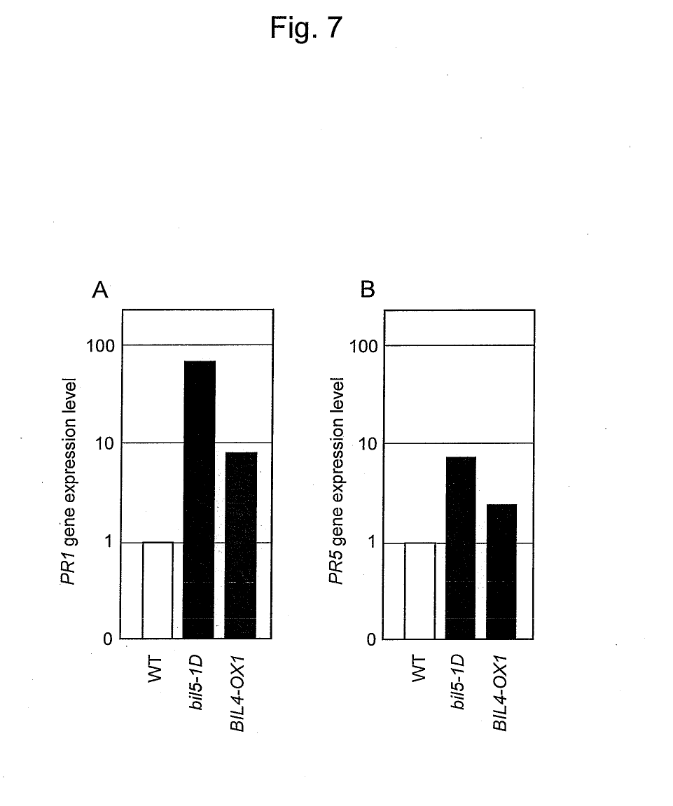

The results are shown in FIG. 7. The expression of the PR1 gene and the PR5 gene was detected in the BIL4-OX1 strain at 9 or more times and 5 or more times, respectively, the levels of the wild-type strain, although these expression levels were smaller than those in the gain-of-function BIL5 mutant bil5-1D. The expression of the pathogen resistance marker genes was also increased in the BIL4 gene-overexpressing strain, as in bil5-1D, suggesting that the BIL4 gene-overexpressing strain is capable of conferring disease resistance to the plant.

### Example 8

### <Induction of PR1 and PR5 Gene Expression in BIL6 Gene-Overexpressing Strain>

Whether the overexpression of the BIL6 gene increased the expression of pathogen resistance marker PR1 and PR5 genes was tested.

### (Method)

Basic procedures therefor followed the method described in Example 2. This Example employed a wild-type strain and two wild-type BIL6 gene-overexpressing transgenic BIL4-OX1 strains (BIL6-OX1 and BIL6-OX2) of Arabidopsis thaliana.

### (Results)

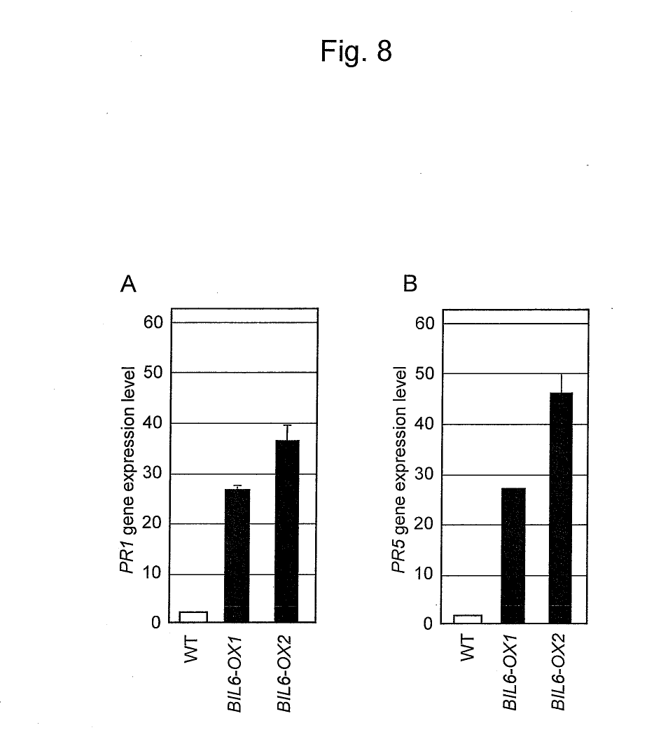

The results are shown in FIG. 8. The expression of the PR1 gene and the PR5 gene was detected in both the BIL6 gene-overexpressing strains at approximately 10 times and approximately 15 to 25 times, respectively, the levels of the wild-type strain. The expression of the pathogen resistance marker genes was also increased in the BIL6 gene-overexpressing strains, suggesting that the BIL6 gene-overexpressing strain is capable of conferring disease resistance to the plant.

All publications, patents, and patent applications cited herein are incorporated herein by reference in their entirety.

1-11. (canceled)

12. A method for conferring disease resistance to a plant, comprising the step of introducing at least one nucleic acid expression system incorporating, in an overexpressible state, a nucleic acid encoding any of the following polypeptides (a) to (c) or a fragment thereof having plant disease resistance-enhancing activity, and/or any of the following polypeptides (d) to (f) or a fragment thereof having plant disease resistance-enhancing activity into a plant of interest:
(a) a polypeptide consisting of the amino acid sequence represented by SEQ ID NO: 1,
(b) a polypeptide consisting of an amino acid sequence derived from the amino acid sequence represented by SEQ ID NO: 1 by the deletion, substitution, or addition of one or several amino acids and having plant disease resistance-enhancing activity,
(c) a polypeptide consisting of an amino acid sequence having 70% or more identity to the amino acid sequence represented by SEQ ID NO: 1 and having plant disease resistance-enhancing activity,
(d) a polypeptide consisting of the amino acid sequence represented by SEQ ID NO: 2,
(e) a polypeptide consisting of an amino acid sequence derived from the amino acid sequence represented by SEQ ID NO: 2 by the deletion, substitution, or addition of one or several amino acids and having plant disease resistance-enhancing activity, and
(f) a polypeptide consisting of an amino acid sequence having 70% or more identity to the amino acid sequence represented by SEQ ID NO: 2 and having plant disease resistance-enhancing activity.

13. The method according to claim 1, wherein the nucleic acid is
a nucleic acid consisting of the nucleotide sequence represented by SEQ ID NO: 3 or 4, or
a nucleic acid that hybridizes under stringent conditions to the nucleic acid consisting of the nucleotide sequence represented by SEQ ID NO: 3 or 4 and encodes a polypeptide having plant disease resistance-enhancing activity.

14. The method according to claim 1, wherein the nucleic acid expression system constitutively expresses the nucleic acid incorporated therein.

15. The method according to claim 1, wherein the nucleic acid expression system inducibly expresses the nucleic acid incorporated therein.

16. The method according to claim 1, wherein the nucleic acid expression system is an expression vector.

17. A disease-resistant plant obtained by a method according to claim 1, or progeny thereof.

18. A disease-resistant plant comprising at least one exogenous nucleic acid expression system incorporating, in an overexpressible state, a nucleic acid encoding any of the following polypeptides (a) to (c) or a fragment thereof having plant disease resistance-enhancing activity, and/or any of the following polypeptides (d) to (f) or a fragment thereof having plant disease resistance-enhancing activity:
(a) a polypeptide consisting of the amino acid sequence represented by SEQ ID NO: 1,
(b) a polypeptide consisting of an amino acid sequence derived from the amino acid sequence represented by SEQ ID NO: 1 by the deletion, substitution, or addition of one or several amino acids and having plant disease resistance-enhancing activity,
(c) a polypeptide consisting of an amino acid sequence having 70% or more identity to the amino acid sequence represented by SEQ ID NO: 1 and having plant disease resistance-enhancing activity,
(d) a polypeptide consisting of the amino acid sequence represented by SEQ ID NO: 2,
(e) a polypeptide consisting of an amino acid sequence derived from the amino acid sequence represented by SEQ ID NO: 2 by the deletion, substitution, or addition of one or several amino acids and having plant disease resistance-enhancing activity, and
(f) a polypeptide consisting of an amino acid sequence having 70% or more identity to the amino acid sequence represented by SEQ ID NO: 2 and having plant disease resistance-enhancing activity.


SEQUENCE LISTING (14 sequences)

SEQ ID NO: 1 - protein, 992 aa, Arabidopsis thaliana
MFDDQAIERGQTISPELTRGIRESRISIVVLSKNYASSSWCLDELLEILKCKEDIGQIVMTVFYGVDPSDVRKQTGDILKVFKKTCSGKTEEKRRRWSQALNDVGNIAGEHFLNWDNESKMMEKIARDISNKVNTTISRDFEDMVGVETHLEKIQSLLHLDNDDEAMIVGIYGPAGIGKTTIARALHSLLSDRFQLTCFMENLRGSYNSSLDEYGLKLQLQEQLLSKILNQTGMRVYNLSAIQGMLCDQKVLIILDDVDDLKQLEALANETKWFGPGSRVVVTTENQELLKQHDDIKNTYYVDFPTQKEARQIFCRYGFKQSTPQDGFENLSERVIKLCSKLPLGLSVMGLYLRKKTEDDWEDILHRLESSFDSVDRNIERVLRVGYDGLHEKDQLLFLLIAFFFNYKDDDHVKAMLADNNLNVRLGLKTLEYKSLIQRSSGGNIVMHKLLQQVGREAVQRQEPWKRQILIDAHEICNVLETDSGCANVMGISFNVSTIPNGVHISAKAFQNMRNLRFLSIYETRRDVNLRVNVPDDMDFPHRLRSLHWEVYPGKSLPSTFRPEYLVELNLQNNKLEKLWEGTQPLTNLNKLELCGSLRLKELPDLSSATNLKRLDLTGCWSLVEIPSSVGNLHKLEELEMNLCLQLQVVPTHFNLASLRSLRMLGCWELRKFPGISTNITSLVIGDAMLEEMLESIRLWSCLETLVVYGSVITHNFWAVTLIEKMGTDIERIPDCIKDLPALKSLYIGGCPKLFSLPELPGSLRRLTVETCESLKTVSFPIDSPIVSFSFPNCFELGEEARRVITQKAGQMIAYLPGREIPAEFVHRAIGDSLTIRSSFCSIFRICVVVSPKSEMKEEYVGFMCRKRINGCPIGDNLFKAQLRKLQAEHLFIFQFEFLEEDGWLEQDNEVLFKFTTSSEELDIIECGIQILTGETNRNISTYGSYESRSEQVSEYEDGYHSDRRLEFHEQKSLPGWGFCGIFHGFLRCFMV

SEQ ID NO: 2 - protein, 1131 aa, Arabidopsis thaliana
MSDLMASPSSSKPHNYKFKVFSSFHGPDVRKTLLSNLREHFQGKGITMFDDEKIKRGGDLSPSLKRAIKTSKISIVILSQKYASSSWCLDELLEIMKRKKAMKQIVMTVFYGVEPSDVRKQTGDFGIAFNKTCVNKTDKERKEWSKALTDVSNIAGEDFKKWDNEANMIKKIARDVSYKLNATPSKDFEDMMGLEAHLKKIQSLLRLDYKDEALIIGISGPAGIGKSTIARALESRLSDRFQLTCFMDLRGSENNGLHDYGQQLRLQEQLLAKVLNQDGTRICHLGVLQQRLSDLRVLIILDDVSDIKQLKALAKETTWFGPGSRIIVTTENKDLLQQRGIDSTYHVGFPSREEALEIFCKFAFEQSSPPHAFEKLAARITHLCGNLPLGLCVMGSSLFGKKQDEWEFVVHRLETNPGQEIDDVLRVGYERLHENDQMLFLHIAIFFNYRDRDLVEAMLADDGNLDVGNWLKFLINKSLIEIYRTGQIVMHKLLQQVGRQAIRRQEPWKRQILINANEICDLLRYEKGTSCNVSGISFDTSGISEVTICDGAFKRLHDLRFLHVYKSRDDGNNRVHIPEKVEFPPRLRLLHWAAYPSKSLPPTFNLECLVELNMRESLVEKLWEGTQHLKNLKYMDLTESKNLKELPDLSNATNLEYFYLDNCESLVEIPSSFAHLHKLEWLEMNNCINLQVIPAHMNLTSVKQVNMKGCSRLRKFPVISRHIEALDISDNTELEDMPASIASWCHLVYLDMSHNEKLQGLTQLPTSLRHLNLSYTDIESIPDCIKALHQLEELCLSGCTRLASLPDLPCSIKALEAEDCESLESVSSPLYTPSARLSFTNCFKLGGEAREAIIRRSSDSTGSVLLPGREVPAEFDHRAQGNSLSILLPLGGNSQFMVCVVISPRHDITKMSNESELLCRINGESCSYDEEFDIVDVSNCRREHLFIFHSGLLRMGRSEAGTEMVFEFSSALQEDFDIIECGVKIWTPQSIRRGYLAFGEDDDGTDGVICDTEDVDQMTDETKGGNNGGSSDEEDGKIHMYSHESGETSESESESEKEDESEPRETSEADADGDYESVSRKRAKKTTSASTDLKLVSIDYLRISNLLKYIFLILVLLLILVVLMSMRLFLA

SEQ ID NO: 3 - DNA, 3241 bp, Arabidopsis thaliana
tggagataccgcgtcttcacgagcttccatggacctgacgtccgtaaaacctttctcagtcatttacgtaaacagtttatctgcaatgggactaccatgttcgatgatcaagcaatcgaaagaggccaaaccatttcccctgaactcacacgagggattagggaatcgaggatctcgatcgtagtgctctcgaagaactatgcttcatcgagctggtgtttggatgagctgttggagattttgaaatgcaaggaagatattgggcaaatagtgatgactgtcttctacggagtagatccatctgatgtgcgcaaacaaaccggagatatcttgaaagttttcaagaaaacttgttctggtaaaacggaggagaagaggcgaagatggagccaagctttgaacgatgttggcaacattgccggagaacatttccttaactgggataatgaatcgaagatgatggagaagattgcaagagatatttcaaacaaagtaaacactacaatctctagggattttgaagacatggtgggagttgagacacacctggagaaaatacagtctttgttacatttagataatgatgatgaagctatgattgttggaatctatggccctgcaggtattggtaagactaccattgccagagctttgcacagtctactctctgataggtttcagcttacttgttttatggagaatcttaggggaagctataatagtagtcttgacgagtatggtttgaagctgcaattacaagaacaacttctttccaagattttaaaccaaactggtatgagggtatacaatttaagtgcaatacagggaatgctatgcgaccaaaaagtgcttatcattcttgatgatgtggacgatctgaagcaactagaagctttggctaatgaaactaagtggtttggtcctgggagtagggttgtagtgaccacagaaaatcaagagcttttgaagcaacatgacgatattaagaatacatactatgtggattttcctactcagaaagaagctcgtcagatcttttgtagatatggttttaaacagagcactccacaagatggtttcgaaaatctttccgaaagagttataaagctttgcagcaaacttccactaggtcttagtgtcatgggtttatatctacgaaaaaagacagaagatgactgggaagatatactgcataggctagaaagtagctttgattcagttgatcgaaatatcgagagagtactaagagtcggatatgacggtttacatgagaaggatcaattgttatttctcctcattgcattcttcttcaactacaaagacgatgatcatgtgaaagcaatgcttgctgacaataacttgaatgtcagactcggtttgaaaactctcgagtataaatctcttatacaaagatcgtctggaggaaatatagtgatgcacaagttactacaacaagtgggtagagaagcggttcaaagacaagagccttggaaacgccaaatcttaattgatgctcatgagatttgtaatgtcctcgaaactgattctggttgtgcaaatgtgatgggcatatcctttaatgtatctacaatcccgaatggcgtgcatataagcgcaaaagcttttcaaaatatgcgtaatcttcgatttctaagcatctacgagacaagacgtgatgtaaaccttagagtaaatgtacccgatgacatggatttcccacatcgtctaaggtctttacattgggaggtatacccaggaaagtctcttccttctacatttaggccagaatatcttgtggaactcaacttgcaaaataacaagctagagaagctttgggaaggaacccagcctcttacaaatctcaacaagttggaattgtgtgggtccttgagattgaaggaacttccagatctttcaagtgctacaaatctcaagagactagatctaactggttgttggagtttggtagagattccttcctctgttggaaatcttcacaaactagaagagttggagatgaatttatgtttacagctacaagttgttccgactcacttcaacttggcatctcttagatcactcaggatgttgggatgctgggaattaaggaaatttccaggtatttctacgaacatcacatccctcgtaatcggagatgcaatgttagaagaaatgcttgaatcaattaggctttggtcttgccttgagactctggttgtatatggcagtgtcattacacataatttttgggcagtaactttaatagagaaaatgggcacagatatagagaggattcctgattgcatcaaagatctccctgcgttaaaatcgctttatataggtggctgtccaaaacttttttcactgccagagctccctggctcactcagaagactaacggtagaaacttgtgaatcactcaagactgtatcttttcctattgattctccaattgtgagtttcagtttccccaactgcttcgaattgggtgaagaagcacggagagtgattacacagaaagcaggtcagatgatagcatacttaccaggaagggaaatacctgcggagttcgtgcacagagctataggagattcgttgaccatccgttcatctttctgctccatatttaggatttgcgttgtggtttcccctaaatcagagatgaaagaagaatatgttggttttatgtgtcgcaaacgcataaacggttgccccattggcgataacttatttaaggcacagctccgtaaactccaagcggaacatctatttatatttcaatttgaattccttgaggaagacggatggctggaacaggacaacgaggtattgttcaaattcacgacctcatccgaggaactagacattattgaatgtggtatccagatattgacaggcgaaaccaacagaaacataagcacctatggaagctatgaatccagatcagagcaagtctctgaatacgaagatggatatcactctgataggagacttgagttccacgaacaaaagtccttgcccgggtggggtttctgtggaatttttcatggcttcttgagatgttttatggtctgacaatgtggctgccgttcatgaaatttctcagaaatggcctagtcaagtgggaccatctctctcttcatcctagccacggtagtcttatcgtggatgttgaatattgctgcgattcagaggaagattgcattgcatccaaagtgtgaaagtcctctcatttcacacc

SEQ ID NO: 4 - DNA, 3514 bp, Arabidopsis thaliana
atttagctacccaaaataacacattaaacttcgaagtatcggatattacttatcttccaaatttcgttacctcaattccatctatctcaggcatgtctgatctcatggcttccccttcttcttccaagcctcacaactacaaattcaaagtcttctcgagcttccacgggcccgacgtccgtaaaacattactcagtaacttgcgcgaacattttcaaggcaaggggataactatgtttgatgacgaaaagattaagagaggcggagacctctctccttcactcaaaagagctataaaaacatcgaagatctcgatcgtgattctctcccagaaatatgcttcttcaagctggtgtttggatgaattgttggagatcatgaagcgcaaaaaggctatgaaacagatagtgatgaccgtcttctacggagtagagccatccgatgtgcggaagcagaccggggatttcgggatcgctttcaataaaacttgtgtgaataagacggacaaggaaagaaaggaatggagcaaagctttgactgatgtgagcaatattgctggagaagacttcaagaaatgggataatgaagcgaacatgatcaagaagattgcaagggatgtttcatataaacttaatgctacaccatcaaaggattttgaagatatgatgggactagaagctcacttgaagaaaatccagtctttgttacgtttagattataaggatgaagctctgataattggaatctctggtcctgcgggcattggtaagagtaccattgctagagctttggagagccgactctctgataggtttcaacttacatgttttatggatcttagaggaagcgagaataatggtcttcacgattacggtcagcagttgcgtttacaagaacaacttcttgcaaaggttttgaatcaagatggtacgaggatatgccatttaggagtgttacaacaaaggctaagcgacctgagagtgcttatcattcttgatgatgtgagtgatataaagcaattaaaggctttggctaaggaaactacttggtttggtcctggaagtaggattattgtgacaaccgaaaacaaagatcttttgcagcaacgtggcatcgatagtacgtaccatgtaggattcccatctagagaagaagctcttgaaatcttctgtaaatttgcttttgaacaaagctctccacctcatgctttcgagaagcttgcagcgagaataacacacttatgtggtaacctcccattgggtctctgtgtgatgggttcatctttatttgggaagaagcaggacgaatgggaatttgtagtgcataggctagaaactaatcctggtcaagaaatcgatgacgtattaagagtcggctatgaaaggttacatgaaaatgatcaaatgctatttctccacattgcaattttcttcaactacagagaccgtgatcttgtggaagccatgcttgctgatgatggtaacttggatgtcggaaactggctgaaattcctaattaacaaatctctgatagagatatatcgcactggacaaatagtgatgcacaagttactacaacaagtgggtagacaagccattcgtagacaggagccttggaaacgacaaatcttaataaatgccaatgagatttgcgatctccttcgatatgagaaaggtactagctgtaatgtgtctggcatatcatttgatacatcaggaatcagtgaagtgactatatgtgatggcgctttcaaaagactgcatgatcttcgatttctccatgtttacaaaagtagagatgatgggaataacagagtgcacatacctgagaaagtggagtttccacctcgtctaaggttactgcattgggcagcatacccaagcaagagtcttcctcctacatttaatctggaatgtcttgtagaactcaatatgcgggaaagcctggtcgagaagctatgggaaggaacccaacaccttaagaatctcaagtatatggatttgactgagtccaagaatttgaaggaactcccagatctttcaaatgctacaaatctggagtatttttatctggataactgtgagagtttggtagagatcccatcctcttttgcgcatctgcataaactagagtggttggagatgaataactgcataaacctacaagtcattccagctcacatgaacttgacgtccgttaagcaagtcaatatgaaaggatgctcaagattaagaaaatttccagttatttcaaggcacatcgaagcactggatatatcagacaacacagagctagaagatatgcctgcatcaattgcgtcttggtgtcatcttgtgtatctcgatatgagccacaatgaaaagctccagggattaacacaactccccacgagtttgagacatctaaacctaagctatactgatattgagagtattccagattgcatcaaagctcttcatcagttagaagagctttgtctatctggctgcacaagactagcatcattgccagatctcccttgttcaatcaaagccctagaggctgaagattgtgaatcactggagagtgtatctagccctttatacactccaagtgcgcgcctcagtttcaccaattgctttaaattgggtggagaagcacgggaagcaataatccgacgatcatcggattctaccgggtcggttctgttaccaggaagagaagtgccagcagagtttgatcacagagcccaaggaaattctttgtccattcttctcccattgggtggtaattcccaatttatggtatgcgtcgtgatttcccctcgccatgatatcacaaaaatgagtaatgagtcggaactgttatgtcgcatcaacggcgaatcatgctcttatgacgaggagttcgatatagttgatgtctcaaactgtcgaagggaacatctatttatatttcactcaggcttgctacgtatgggccgttctgaagcaggcacagagatggtgttcgaattcagcagcgcattacaagaagacttcgacattattgaatgtggtgtcaagatctggaccccacaaagcatcagacgaggatacctagcgtttggagaagacgatgacggaacagatggagtaatatgtgacacggaagacgtcgatcagatgacggacgaaaccaagggaggcaacaatggaggatctagcgatgaagaggatggcaaaatccacatgtattcacatgaatccggagaaacgtctgaatctgaatctgaatctgagaaagaagacgaatctgaacctagagaaacgtccgaggcagacgcggacggggactacgagtctgtatccaggaagcgtgcgaagaagacgacgagtgctagtacagatcttaagttggtttctatagactacttgcggatttcaaatcttttaaaatatattttccttattttagtattgcttttaatccttgttgtacttatgtctatgcgactttttcttgcttgaaagcatgcggtctttgttttatgcat

SEQ ID NO: 5 - DNA, 18 bp, Artificial
  primer
gagcggtaggcgtaggtc

SEQ ID NO: 6 - DNA, 20 bp, Artificial
  primer
gcccaccagagtgtatgagt

SEQ ID NO: 7 - DNA, 33 bp, Artificial
  primer
caccatgttcgatgatcaagcaatcgaaagagg

SEQ ID NO: 8 - DNA, 28 bp, Artificial
  primer
gtgtgaaatgagaggactttcacacttt

SEQ ID NO: 9 - DNA, 31 bp, Artificial
  primer
caccatgtctgatctcatggcttccccttct

SEQ ID NO: 10 - DNA, 30 bp, Artificial
  primer
tcaagcaagaaaaagtcgcatagacataag

SEQ ID NO: 11 - protein, 239 aa, Oryza sativa
MWNQKHDLESAQTPLYPMMSESPELRWSFIRKVYSIISIQLLVTIAVAATVVKVHSISVFFTTTTAGFALYILLILTPLIVMCPLYYYHQKHPVNYLLLGIFTVALAFAVGLTCAFTSGKVILESVILTAVVVISLTLYTFWAAKRGHDFNFLGPFLFGAVIVLMVFSFIQILFPLGKISVMIYGCLASIIFCGYIVYDTDNLIKRHSYDEYIWAAVSLYLDVINLFLSLLTLLRAVDS

SEQ ID NO: 12 - protein, 456 aa, Medicago truncatula
MSDLLGHLHKFEQEFVFMCKTKMGVNASEPKRTRSRPSQSQGRHPSTRRSPSTSNTGTTTTTTTSNSNKTGASSSSSGAASSSVASRTSLASLRESLPENPHIYNVSEIRAATNNFLANRLSSSSSKASWRCNLRGKEVVVFQRKFRRRIDTDELRDRLSDICRSHHGSIINLLGASVSGGGRVGGGGGGGGDHIYLVYEYVNGASLADCLRNPKNPKFTVLSNWTSRIQIATDLAHGLDYIHNKTGLKIENLVHNHIKSSAVIVTEPDFNAKICHFGTAQLCGETDEMSLERDESRNSRRSDSRAIRFEGVRGYMSPEFQNLGIATQKSDVYAFGVMMLELLSGEEPLKYRYDKATGDFERTSVIETARAAIEDGDGDRDGRLRRWMDRRLEDSFPVTVVEKLTRLALDCVQDDPVNRPEMGRVAGKISQLYLESEKWSANMKRPTDITVSFAPR

SEQ ID NO: 13 - DNA, 720 bp, Oryza sativa
atgtggaatcagaagcatgatttggagtcggctcagacgcctctctacccgatgatgtcggagagcccggagctccgatggtctttcatccgcaaggtctattccatcatctccatccagctgctcgttacaatcgccgtcgctgccaccgtcgttaaggttcattctatttctgtattcttcaccaccaccaccgccggcttcgctctctacatacttcttatattgactccattgatcgttatgtgtcctttgtattactatcaccagaagcaccctgtgaactatctgttactcgggattttcaccgtcgctcttgcctttgcggttggcttgacctgcgcctttacaagcgggaaggtgattctggaatctgtgattttgaccgctgttgtggtcatatccctcactctatacactttctgggcggccaaaagaggccatgacttcaacttcctcggacctttcttgttcggtgctgtcattgtcctcatggtgttttccttcatccagattctcttccctttaggaaagatctctgtgatgatctatggttgtttggcatccatcatcttctgtggctacattgtatatgataccgacaacttgatcaagaggcactcctatgacgagtacatctgggctgctgtttctctttatttggatgtcattaatctcttcctatctctcctcactctcttaagggctgttgatagttag

SEQ ID NO: 14 - DNA, 1371 bp, Medicago truncatula
atgtctgatcttctaggtcacctccacaaattcgaacaagaatttgtctttatgtgcaaaaccaaaatgggagtcaatgcttcagagccaaaacgaacacgatcgcgaccaagtcaatcgcaggggaggcatccgtcgacgagacgatctccgtcaacgagcaacaccggcacaacaacaaccacgacgacatcgaattctaacaaaactggtgcctcctcttcctcctccggtgcggcttcttcctcagtcgctagtcgaacatctctcgctagtctccgagaatccttaccggaaaatcctcacatatacaacgtctccgagattcgtgccgctaccaacaatttcctcgctaatcgcttatcttcctcttcctctaaagcctcctggcgttgcaatttaagagggaaagaagtcgttgtgtttcagagaaagtttaggcgaaggatcgacacggatgagcttagagatcgtctttcagatatttgccggagccaccacggaagcatcattaatctactcggcgcttctgtttccggcggtggaagagtaggaggaggaggaggaggaggaggtgatcacatctacttggtctacgaatacgtcaacggagcatctctagctgattgcttgagaaaccctaagaaccctaaatttacagttctatcgaattggacttcacggattcaaattgcaacagatctagctcatggactcgattacatccacaacaaaaccggattaaagatcgaaaacctagttcacaatcacatcaagagctcggcggtgatcgtgacggagccagatttcaacgctaagatctgtcacttcggaactgcgcagctttgcggcgaaacagatgaaatgtcactggagagagacgaatcgagaaattcaaggcgatctgatagcagagcgatcagattcgaaggagttagaggttacatgtcgccggaatttcaaaatttgggaatcgctactcagaaatcggatgtttacgctttcggagttatgatgttggagcttctctcaggcgaggagccgttgaagtacaggtatgataaagccacaggagatttcgagcggacgtcggtgattgaaacggcgagagcggcgatcgaagatggagatggtgatagagatggaagactgaggaggtggatggatcggagattggaggattcgtttcctgtgacggtggtggagaagttaacgaggctagcgttggattgtgttcaggatgatccagtgaataggccggagatgggaagagtcgccggaaaaatatcacaactgtatttagaatcggaaaaatggtcggcgaatatgaaacggccaacggatatcaccgtctctttcgctcccagatga